(19) United States
(12) Patent Application Publication
(10) Pub. No.: US 2022/0112225 A1
(43) Pub. Date: Apr. 14, 2022

(54) ANANDAMIDE COMPOUNDS

(71) Applicant: TRAVECTA THERAPEUTICS PTE. LTD., Singapore (SG)

(72) Inventors: David Lawrence Silver, Singapore (SG); Mahmood Ahmed, Singapore (SG); Sing Yeung Frankie Mak, Singapore (SG)

(21) Appl. No.: 17/421,924

(22) PCT Filed: Jan. 10, 2020
(86) PCT No.: PCT/US2020/013150

§ 371 Date: Jul. 9, 2021

Related U.S. Application Data
(60) Provisional application No. 62/790,787, filed on Jan. 10, 2019.

(51) Int. Cl. C07F 9/09; C07C 233/20

(52) U.S. Cl. CPC C07F 9/091; C07C 233/20

(57) ABSTRACT

The present application provides anandamide and 2-arachidonoyl glycerol compounds useful for treating a disease or disorder in a subject in need thereof. Pharmaceutical compositions comprising the compounds and methods of treating diseases or disorders are also provided.

### CROSS-REFERENCE TO RELATED APPLICATIONS

This application claims the benefit of U.S. Provisional Application Ser. No. 62/790,787, filed Jan. 10, 2019, the disclosure of which is incorporated herein by reference in its entirety.

### TECHNICAL FIELD

The present application provides anandamide and 2-arachidonoyl glycerol compounds useful for treating a disease or disorder in a subject in need thereof.

### BACKGROUND

The endocannabinoid (eCB) system has been implicated in a variety of physiological processes including cell signalling, memory encoding, compensatory mechanisms, and immunosuppressant and anti-inflammatory responses. The eCB system comprises at least two receptors: the CB1 cannabinoid receptor, widely distributed in the brain and present in some peripheral organs, and the CB2 receptor, found principally in the periphery and immune systems and in some regions of the brain. The endogenous agonists of these receptors are the endogenous cannabinoids (eCBs), a family of lipids comprising anandamide (AEA) and 2-arachidonoyl glycerol (2-AG) as well as other closely related compounds (see e.g., Piomelli, Nat. Rev. Neurosci. 2003, 4(11), 873).

### SUMMARY

The present invention relates to, inter alia, compounds of Formula I:

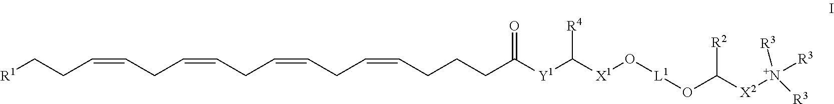

or pharmaceutically acceptable salts, wherein constituent members are defined herein.

The present invention further provides pharmaceutical compositions comprising a compound of Formula I, or a pharmaceutically acceptable salt thereof, and a pharmaceutically acceptable carrier.

The present invention further provides methods of treating a disease or disorder in a subject, comprising administering to a subject a therapeutically effective amount of a compound of Formula I, or a pharmaceutically acceptable salt thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF DRAWINGS

FIGS. 1A-1B show phospholipid separation from hMfsd2a cells (WT), D97A transfected HEK293 cells (D97A), and empty vector transfected cells (EV) treated with Compound 3 (anandamide PC) by thin layer chromatography (TLC) via iodine stain (FIG. 1A) and cupric acetate stain (FIG. 1B).

FIGS. 2A-2B show phospholipid separation from hMfsd2a cells (WT), D97A transfected HEK293 cells (D97A), and empty vector transfected cells (EV) treated with Compound 4 by thin layer chromatography (TLC) via iodine stain (FIG. 2A) and cupric acetate stain (FIG. 2B).

FIGS. 3A-3B show phospholipid separation from hMfsd2a cells (WT), D97A transfected HEK293 cells (D97A), and empty vector transfected cells (EV) treated with Compound 5 by thin layer chromatography (TLC) via iodine stain (FIG. 3A) and cupric acetate stain (FIG. 3B).

FIGS. 4A-4B show phospholipid separation from hMfsd2a cells (WT), D97A transfected HEK293 cells (D97A), and empty vector transfected cells (EV) treated with Compound 6 by thin layer chromatography (TLC) via iodine stain (FIG. 4A) and cupric acetate stain (FIG. 4B).

FIGS. 5A-5B show phospholipid separation from hMfsd2a cells (WT), D97A transfected HEK293 cells (D97A), and empty vector transfected cells (EV) treated with Compound 7 by thin layer chromatography (TLC) via iodine stain (FIG. 5A) and cupric acetate stain (FIG. 5B).

FIGS. 6A-6B show phospholipid separation from hMfsd2a cells (WT), D97A transfected HEK293 cells (D97A), and empty vector transfected cells (EV) treated with Compound 8 by thin layer chromatography (TLC) via iodine stain (FIG. 6A) and cupric acetate stain (FIG. 6B).

FIGS. 7A-7B show phospholipid separation from hMfsd2a cells (WT), D97A transfected HEK293 cells (D97A), and empty vector transfected cells (EV) treated with Compound 20 by thin layer chromatography (TLC) via iodine stain (FIG. 7A) and cupric acetate stain (FIG. 7B).

FIGS. 8A-8G show results of the in vitro MFSD2A transport assay in hMfsd2a cells (WT), D97A transfected HEK293 cells (D97A), and empty vector transfected cells (EV) treated with Compound 3 (FIG. 8A), Compound 4 (FIG. 8B), Compound 5 (FIG. 8C), Compound 6 (FIG. 8D), Compound 7 (FIG. 8E), Compound 8 (FIG. 8F), or Compound 20 (FIG. 8G).

FIG. 9A shows the amount, of Compound 3 remaining in the brain homogenate described in Example 11.

FIG. 9B shows the amount of Compound 2 formed in the brain homogenate treated with Compound 3, as described in Example 11.

FIG. 9C show's the amount of Compound 4 remaining in the brain homogenate described in Example 11.

FIG. 9D show's the amount of Compound 2 formed in the brain homogenate treated with Compound 4, as described in Example 11.

FIG. 9E shows the amount of Compound 5 remaining in the brain homogenate described in Example 11.

FIG. 9F show's the amount of Compound 2 formed in the brain homogenate treated with Compound 5, as described in Example 11.

### DETAILED DESCRIPTION

The magnitude and duration of in vivo CB1 and/or CB2 receptor modulation by AEA and 2-AG is relatively short, presumably due to rapid inactivation process involving endocannabinoid deactivating proteins, with AEA and 2-AG predominantly hydrolyzed by Fatty Acid Amide Hydrolase (FAAH) and monoacylglycerol lipase (MAGL), respectively. FAAH and MAGL are serine hydrolase and their inhibition is known to increase the level of endogenous cannabinoid ligands, including AEA and 2-AG. The increased level of activation of the cannabinoid receptors resulting from increased level of AEA and/or 2-AG has shown analgesic effect in acute and chronic models of pain, as well as a number of other animal models (e.g., depression, anxiety, inflammation, brain trauma, multiple sclerosis, cancer, and glaucoma) (see e.g., Nomura, Life Sci. 2013, 92(8-9), 492; and Mallet, Int. J. Clin. Pharmacol Ther. 2016; 54(7), 498).

Accordingly, the present application provides an alternative approach to increase the levels of endogenous cannabinoid ligands via the administration of the compounds described herein.

### Compounds

The present application provides, inter alia, a compound of Formula I:

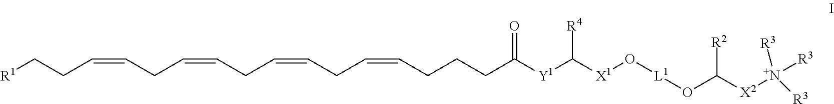

or a pharmaceutically acceptable salt thereof, wherein:

L¹ is CO or PO₂;

X¹ is selected from the group consisting of C₁₋₄ alkylene, C₁₋₄ alkylene-OC(O)O—C₁₋₄ alkylene, C₁₋₄ alkylene-OC(O)C₁₋₄ alkylene, C₁₋₄ alkylene-O—C₁₋₄ alkylene, C₁₋₄ alkylene-O—C₁₋₄ alkylene-O—C₁₋₄ alkylene, and C₁₋₄ alkylene-O—C₁₋₄ alkylene-O—C(O)C₁₋₄ alkylene, wherein each C₁₋₄ alkylene is optionally substituted by OH or CO₂;

X² is C₁₋₆ alkylene, which is optionally substituted by OH or CO₂;

Y¹ is selected from the group consisting of O, S, and NR⁵;

R¹ is C₁₋₁₀ alkyl;

R² is selected from the group consisting of H and C₁₋₆ alkyl, wherein the C₁₋₆ alkyl is optionally substituted by OH or CO₂;

each R³ is independently selected from the group consisting of H and C₁₋₆ alkyl;

R⁴ is selected from the group consisting of H and C₁₋₆ alkyl, wherein the C₁₋₆ alkyl is optionally substituted by OH or CO₂; and

R⁵ is selected from the group consisting of H and C₁₋₆ alkyl, wherein the C₁₋₆ alkyl is optionally substituted by OH or CO₂.

In some embodiments, the compound of Formula I is not a compound selected from:

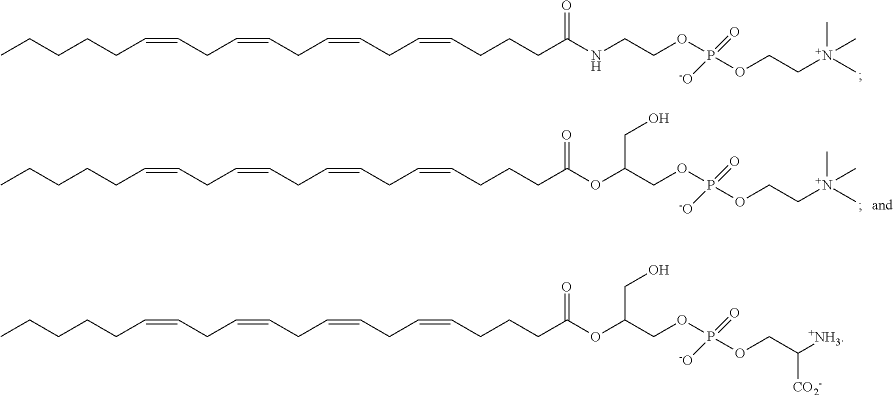

In some embodiments, the compound of Formula I is a compound of Formula VI:

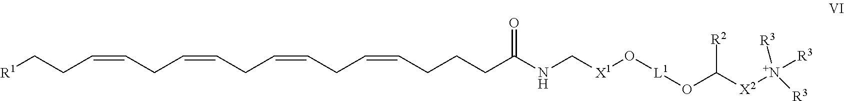

or a pharmaceutically acceptable salt thereof, wherein:

L¹ is CO or PO₂;

X¹ is selected from the group consisting of C₁₋₄ alkylene, C₁₋₄ alkylene-OC(O)O—C₁₋₄ alkylene, C₁₋₄ alkylene-OC(O)C₁₋₄ alkylene, C₁₋₄ alkylene-O—C₁₋₄ alkylene, C₁₋₄ alkylene-O—C₁₋₄ alkylene-O—C₁₋₄ alkylene, C₁₋₄ alkylene-O—C₁₋₄ alkylene-O—C(O)C₁₋₄ alkylene, and C₁₋₄ alkylene-OC(O)NH—C₁₋₄ alkylene, wherein each C₁₋₄ alkylene is optionally substituted by OH or CO₂;

X² is C₁₋₆ alkylene, which is optionally substituted by OH or CO₂;

R¹ is C₁₋₁₀ alkyl;

R² is selected from the group consisting of H and C₁₋₆ alkyl, wherein the C₁₋₆ alkyl is optionally substituted by OH or CO₂; and

each R³ is independently selected from the group consisting of H and C₁₋₆ alkyl.

In some embodiments, the compound of Formula I or Formula VI is a compound of Formula VIa:

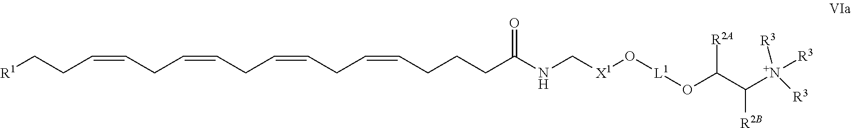

or a pharmaceutically acceptable salt thereof, wherein:

L¹ is CO or PO₂;

X¹ is selected from the group consisting of C₁₋₄ alkylene, C₁₋₄ alkylene-OC(O)O—C₁₋₄ alkylene, C₁₋₄ alkylene-OC(O)C₁₋₄ alkylene, C₁₋₄ alkylene-O—C₁₋₄ alkylene, C₁₋₄ alkylene-O—C₁₋₄ alkylene-O—C₁₋₄ alkylene, C₁₋₄ alkylene-O—C₁₋₄ alkylene-O—C(O)C₁₋₄ alkylene, and C₁₋₄ alkylene-OC(O)NH—C₁₋₄ alkylene, wherein each C₁₋₄ alkylene is optionally substituted by OH or CO₂;

X² is C₁₋₆ alkylene, which is optionally substituted by OH or CO₂;

R¹ is C₁₋₁₀ alkyl;

R^{2A} is H or CH₂CO₂;

R^{2B} is H or CO₂; and

each R³ is independently selected from the group consisting of H and C₁₋₆ alkyl.

In some embodiments, the compound of Formula I is a compound of Formula VII:

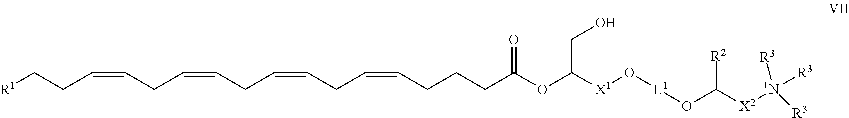

or a pharmaceutically acceptable salt thereof, wherein:

L¹ is CO or PO₂;

X¹ is selected from the group consisting of C₁₋₄ alkylene. C₁₋₄ alkylene-OC(O)O—C₁₋₄ alkylene, C₁₋₄ alkylene-OC(O)C₁₋₄ alkylene, C₁₋₄ alkylene-O—C₁₋₄ alkylene, C₁₋₄ alkylene-O—C₁₋₄ alkylene-O—C₁₋₄ alkylene, C₁₋₄ alkylene-O—C₁₋₄ alkylene-O—C(O)C₁₋₄ alkylene, and C₁₋₄ alkylene-OC(O)NH—C₁₋₄ alkylene, wherein each C₁₋₄ alkylene is optionally substituted by OH or CO₂;

X² is C₁₋₆ alkylene, which is optionally substituted by OH or CO₂;

R¹ is C₁₋₁₀ alkyl;

R² is selected from the group consisting of H and C₁₋₆ alkyl, wherein the C₁₋₆ alkyl is optionally substituted by OH or CO₂; and

each R³ is independently selected from the group consisting of H and C₁₋₆ alkyl.

In some embodiments, the compound of Formula I or Formula VII is a compound of Formula VIIa:

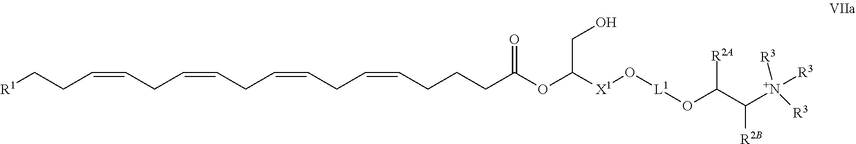

or a pharmaceutically acceptable salt thereof, wherein:

L¹ is CO or PO₂;

X¹ is selected from the group consisting of C₁₋₄ alkylene, C₁₋₄ alkylene-OC(O)O—C₁₋₄ alkylene, C₁₋₄ alkylene-OC(O)C₁₋₄ alkylene, C₁₋₄ alkylene-O—C₁₋₄ alkylene, C₁₋₄ alkylene-O—C₁₋₄ alkylene-O—C₁₋₄ alkylene, C₁₋₄ alkylene-O—C₁₋₄ alkylene-O—C(O)C₁₋₄ alkylene, and C₁₋₄ alkylene-OC(O)NH—C₁₋₄ alkylene, wherein each C₁₋₄ alkylene is optionally substituted by OH or CO₂;

X² is C₁₋₄ alkylene, which is optionally substituted by OH or CO₂;

R¹ is C₁₋₁₀ alkyl;

R^{2A} is H or CH₂CO₂;

R^{2B} is H or CO₂; and

each R³ is independently selected from the group consisting of H and C₁₋₆ alkyl.

In some embodiments, the compound of Formula I is a compound of Formula Ia:

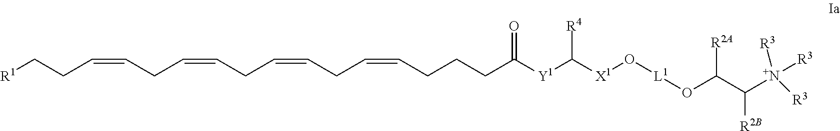

or a pharmaceutically acceptable salt thereof, wherein:

L¹ is CO or PO₂;

X¹ is selected from the group consisting of C₁₋₄ alkylene, C₁₋₄ alkylene-OC(O)O—C₁₋₄ alkylene, C₁₋₄ alkylene-OC(O)C₁₋₄ alkylene, C₁₋₄ alkylene-O—C₁₋₄ alkylene, C₁₋₄ alkylene-O—C₁₋₄ alkylene-O—C₁₋₄ alkylene, and C₁₋₄ alkylene-G-C₁₋₄ alkylene-O—C(O)C₁₋₄ alkylene, wherein each C₁₋₄ alkylene is optionally substituted by OH or CO₂;

X² is C₁₋₆ alkylene, which is optionally substituted by OH or CO₂;

Y¹ is selected from the group consisting of O, S, and NR⁵;

R¹ is C₁₋₁₀ alkyl;

R^{2A} is H or CH₂CO₂;

R^{2B} is H or CO₂;

each R³ is independently selected from the group consisting of H and C₁₋₆ alkyl;

R⁴ is selected from the group consisting of H and C₁₋₆ alkyl, wherein the C₁₋₆ alkyl is optionally substituted by OH or CO₂; and

R⁵ is selected from the group consisting of H and C₁₋₆ alkyl, wherein the C₁₋₆ alkyl is optionally substituted by OH or CO₂.

In some embodiments, the compound of Formula I, Formula Ia, Formula VII, or Formula VIIa is not:

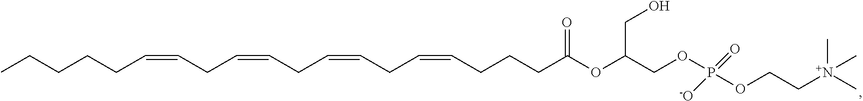

or a pharmaceutically acceptable salt thereof.

In some embodiments, the compound is a compound of Formula Ia, or a pharmaceutically acceptable salt thereof, provided that when R^{2A} is H, then R^{2B} is CO₂ and R⁴ is not hydroxymethyl.

In some embodiments, L¹ is CO. In some embodiments of Formulas I-Ia, VI-VIa, and VII-VIIa, L¹ is PO₂.

In some embodiments, X¹ is selected from the group consisting of CH₂, CH₂OC(O)O—C₁₋₄ alkylene, CH₂OC(O)C₁₋₄ alkylene, CH₂O—C₁₋₄ alkylene, CH₂O—C₁₋₄ alkylene-O—C₁₋₄ alkylene, and CH₂—O—C₁₋₄ alkylene-O—C(O)C₁₋₄ alkylene, wherein each C₁₋₄ alkylene is optionally substituted by OH.

In some embodiments, X¹ is selected from the group consisting of C₁₋₄ alkylene, C₁₋₄ alkylene-OC(O)O—C₁₋₄ alkylene, C₁₋₄ alkylene-OC(O)C₁₋₄ alkylene, C₁₋₄ alkylene-O—C₁₋₄ alkylene, C₁₋₄ alkylene-O—C₁₋₄ alkylene-O—C₁₋₄ alkylene, and C₁₋₄ alkylene-O—C₁₋₄ alkylene-O—C(O)C₁₋₄ alkylene, wherein each C₁₋₄ alkylene is optionally substituted by OH or CO₂.

In some embodiments, X¹ is selected from the group consisting of CH₂, CH₂OC(O)O—C₁₋₄ alkylene, CH₂OC(O)C₁₋₄ alkylene, CH₂O—C₁₋₄ alkylene, CH₂O—C₁₋₄ alkylene-O—C₁₋₄ alkylene, CH₂—O—C₁₋₄ alkylene-O—C(O)C₁₋₄ alkylene, and C₁₋₄ alkylene-OC(O)NH—C₁₋₄ alkylene, wherein each C₁₋₄ alkylene is optionally substituted by OH.

In some embodiments, X¹ is selected from the group consisting of CH₂, CH₂OC(O)OCH₂CH₂, CH₂OC(O)OCH₂CH(OH)CH₂, CH₂OC(O)CH₂CH₂CH₂, CH₂OC(O)CH₂CH₂, CH₂OCH(CH₃), CH₂OCH(CH₃)OCH₂CH(OH)CH₂, CH₂OCH(CH₃)OCH₂CH₂, CH₂OCH(CH₃)OC(O)CH₂, and CH₂OC(O)NHCH₂CH₂.

In some embodiments, X¹ is selected from the group consisting of CH₂, CH₂OC(O)OCH₂CH₂, CH₂OC(O)OCH₂CH(OH)CH₂, CH₂OC(O)CH₂CH₂CH₂, CH₂OC(O)CH₂CH₂, CH₂OCH(CH₃), CH₂OCH(CH₃)OCH₂(OH)CH₂, CH₂OCH(CH₃)OCH₂CH₂, and CH₂OCH(CH₃)OC(O)CH₂. In some embodiments, X¹ is CH₂OC(O)OCH₂CH₂, CH₂OC(O)OCH₂CH(OH)CH₂, CH₂OC(O)CH₂CH₂CH₂, CH₂OC(O)CH₂CH₂, CH₂OCH(CH₃), CH₂OCH(CH₃)OCH₂CH(OH)CH₂, CH₂OCH(CH₃)OCH₂CH₂, and CH₂OCH(CH₃)OC(O)CH₂. In some embodiments, X¹ is CH₂.

In some embodiments, X² is C₁₋₃ alkylene which is optionally substituted with OH or CO₂. In some embodiments, X² is C₁₋₃ alkylene which is optionally substituted CO₂. In some embodiments, X² is selected from the group consisting of CH₂, CHCH₃, and CH₂CO₂. In some embodiments, X² is CH₂ or CH₂CO₂.

In some embodiments, Y¹ is O or NR⁵. In some embodiments, Y¹ is NR⁵. In some embodiments, R⁵ is H or C₁₋₃ alkyl. In some embodiments, Y¹ is NH. In some embodiments, Y¹ is O.

In some embodiments, R¹ is C₁₋₆ alkyl. In some embodiments, R¹ is C₁₋₃ alkyl. In some embodiments, R¹ is propyl.

In some embodiments, R² is selected from the group consisting of H and C₁₋₃ alkyl which is optionally substituted by OH or CO₂. In some embodiments, R² is selected from the group consisting of H and C₁₋₃ alkyl which is optionally substituted by CO₂. In some embodiments, R² is selected from the group consisting of H, CHCH₃, and CH₂CO₂. In some embodiments, R² is H or CH₂CO₂.

In some embodiments, each R³ is independently selected from the group consisting of H and C₁₋₃ alkyl. In some embodiments, each R³ is H. In some embodiments, each R³ is an independently selected C₁₋₃ alkyl group. In some embodiments, each R³ is a C₁₋₃ alkyl group, wherein each R³ is group is the same. In some embodiments, each R³ is methyl or ethyl. In some embodiments, each R³ is methyl. In some embodiments, each R³ is ethyl.

In some embodiments, R⁴ is selected from the group consisting of H and C₁₋₃ alkyl which is optionally substituted by OH or CO₂. In some embodiments, R⁴ is selected from the group consisting of H and C₁₋₃ alkyl which is optionally substituted by OH. In some embodiments, R⁴ is H or hydroxymethyl.

In some embodiments:

L¹ is CO or PO₂;

X¹ is C₁₋₃ alkylene, which is optionally substituted by OH or CO₂;

X² is C₁₋₃ alkylene, which is optionally substituted by OH or CO₂;

Y¹ is O or NR⁵;

R¹ is C₁₋₆ alkyl;

R² is selected from the group consisting of H and C₁₋₃ alkyl which is optionally substituted by OH or CO₂;

each R³ is independently selected from the group consisting of H and C₁₋₃ alkyl;

R⁴ is selected from the group consisting of H and C₁₋₃ alkyl which is optionally substituted by OH or CO₂; and

R⁵ is selected from the group consisting of H or C₁₋₃ alkyl.

In some embodiments:

L¹ is CO or PO₂;

X¹ is C₁₋₄ alkylene;

X² is C₁₋₃ alkylene, which is optionally substituted by OH or CO₂;

Y¹ is O or NH;

R¹ is C₁₋₆ alkyl;

R² is selected from the group consisting of H and C₁₋₃ alkyl which is optionally substituted by CO₂;

each R³ is independently selected from the group consisting of H and C₁₋₃ alkyl;

R⁴ is selected from the group consisting of H and C₁₋₃ alkyl which is optionally substituted by OH.

In some embodiments:

L¹ is PO₂;

X¹ is C₁₋₄ alkylene;

X² is C₁₋₃ alkylene, which is optionally substituted by OH or CO₂;

Y¹ is NH;

R¹ is C₁₋₆ alkyl;

R² is selected from the group consisting of H and C₁₋₃ alkyl which is optionally substituted by CO₂;

each R³ is independently selected from the group consisting of H and C₁₋₃ alkyl;

R⁴ is selected from the group consisting of H and C₁₋₃ alkyl which is optionally substituted by OH.

In some embodiments:

L¹ is CO;

X¹ is C₁₋₄ alkylene;

X² is C₁₋₃ alkylene, which is optionally substituted by OH or CO₂;

Y¹ is O;

R¹ is C₁₋₆ alkyl;

R² is selected from the group consisting of H and C₁₋₃ alkyl which is optionally substituted by CO₂;

each R³ is independently selected from the group consisting of H and C₁₋₃ alkyl;

R⁴ is selected from the group consisting of H and C₁₋₃ alkyl which is optionally substituted by OH.

In some embodiments:

L¹ is CO or PO₂;

X¹ is C₁₋₃ alkylene, which is optionally substituted by OH or CO₂;

X² is C₁₋₃ alkylene, which is optionally substituted by OH or CO₂;

R¹ is C₁₋₆ alkyl;

R² is selected from the group consisting of H and C₁₋₃ alkyl which is optionally substituted by OH or CO₂;

each R³ is independently selected from the group consisting of H and C₁₋₃ alkyl.

In some embodiments:

L¹ is CO or PO₂;

X¹ is C₁₋₄ alkylene;

X² is C₁₋₃ alkylene, which is optionally substituted by OH or CO₂;

R¹ is C₁₋₆ alkyl;

R² is selected from the group consisting of H and C₁₋₃ alkyl which is optionally substituted by CO₂;

each R³ is independently selected from the group consisting of H and C₁₋₃ alkyl.

In some embodiments of Formula VI or Formula VIa:

L¹ is PO₂;

X¹ is C₁₋₄ alkylene;

X² is C₁₋₃ alkylene, which is optionally substituted by OH or CO₂;

R¹ is C₁₋₆ alkyl;

R² is selected from the group consisting of H and C₁₋₃ alkyl which is optionally substituted by CO₂;

each R³ is independently selected from the group consisting of H and C₁₋₃ alkyl.

In some embodiments of Formula VII or Formula VIIa:

L¹ is CO;

X¹ is C₁₋₄ alkylene;

X² is C₁₋₃ alkylene, which is optionally substituted by OH or CO₂;

R¹ is C₁₋₆ alkyl;

R² is selected from the group consisting of H and C₁₋₃ alkyl which is optionally substituted by CO₂;

each R³ is independently selected from the group consisting of H and C₁₋₃ alkyl.

In some embodiments, the compound of Formula I or Formula VI is a compound of Formula II:

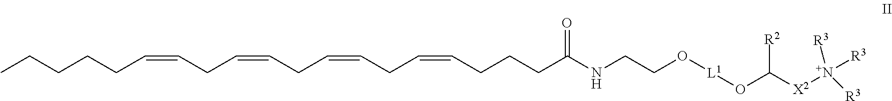

or a pharmaceutically acceptable salt thereof wherein variables L¹, X², R², and R³ are defined according to the definitions provided herein for compounds of Formula I and Formula VI.

In some embodiments, the compound of Formula I or Formula VI is a compound of Formula III:

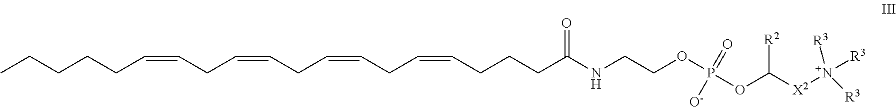

or a pharmaceutically acceptable salt thereof, wherein variables X², R², and R³ are defined according to the definitions provided herein for compounds of Formula I and Formula VI.

In some embodiments, the compound of Formula I or Formula VII is a compound of Formula IV:

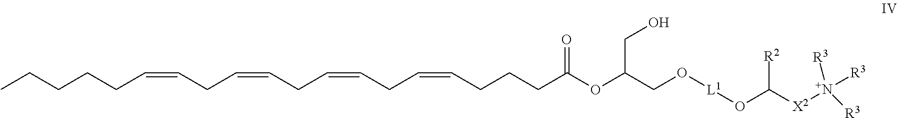

or a pharmaceutically acceptable salt thereof, wherein variables L¹, X², R², and R¹ are defined according to the definitions provided herein for compounds of Formula I and Formula VII.

In some embodiments, the compound of Formula I or Formula VII is a compound of Formula V:

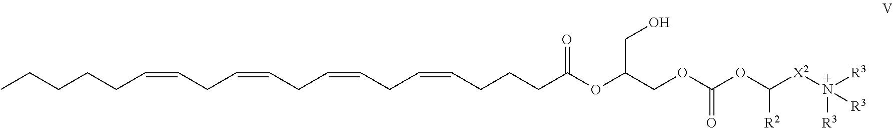

or a pharmaceutically acceptable salt thereof, wherein variables X², R², and R³ are defined according to the definitions provided herein for compounds of Formula I and Formula VII

In some embodiments, the compound of Formula I is selected from the group consisting of:

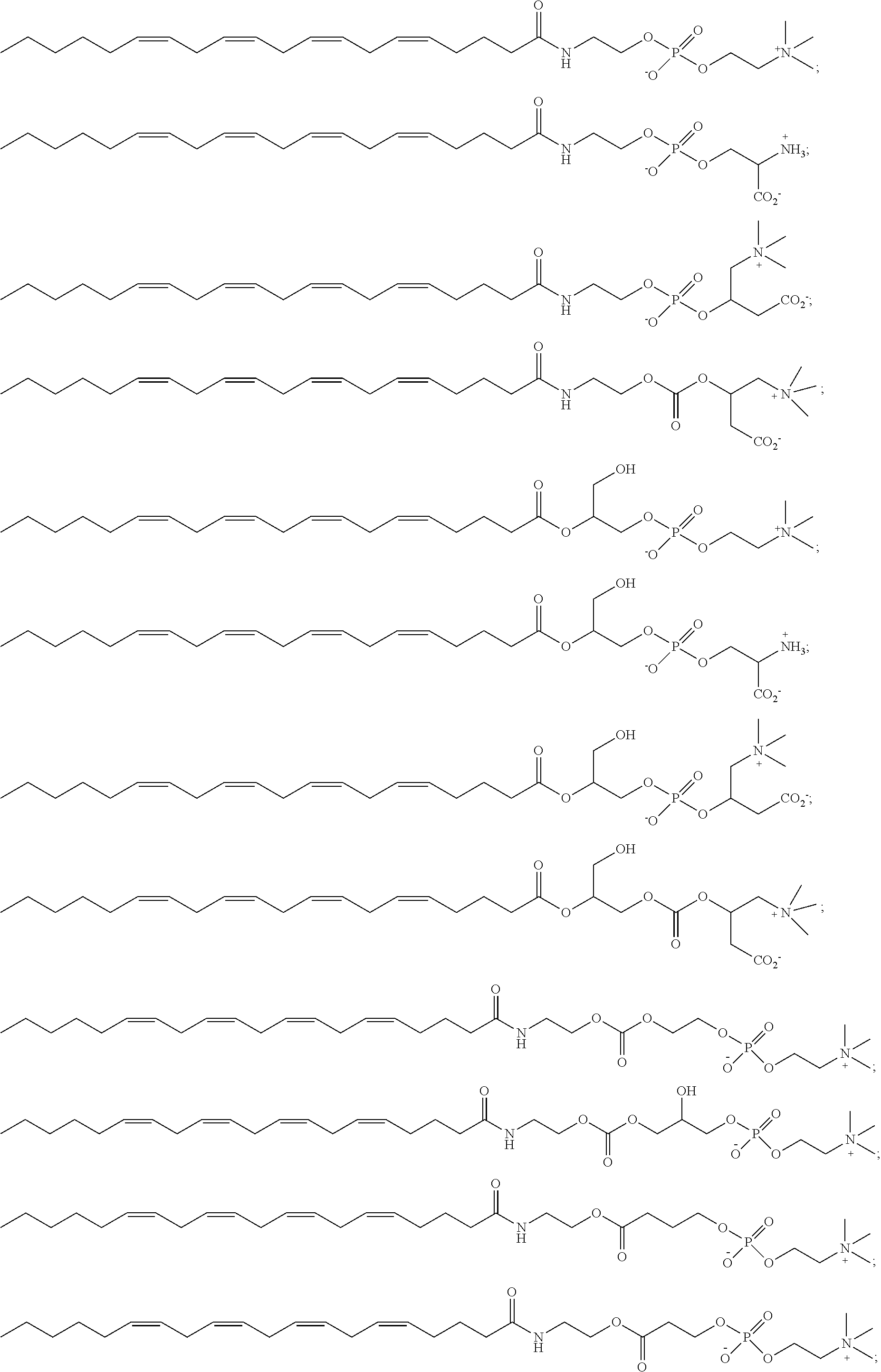

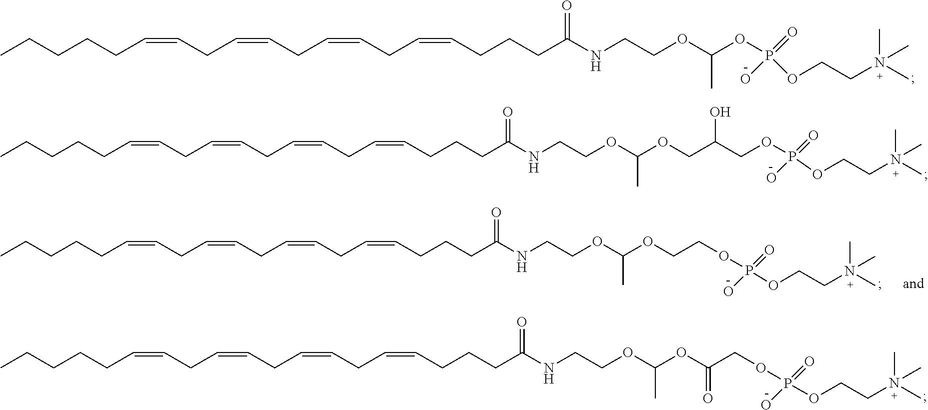

or a pharmaceutically acceptable salt thereof.

In some embodiments, the compound of Formula I is selected from the group consisting of:

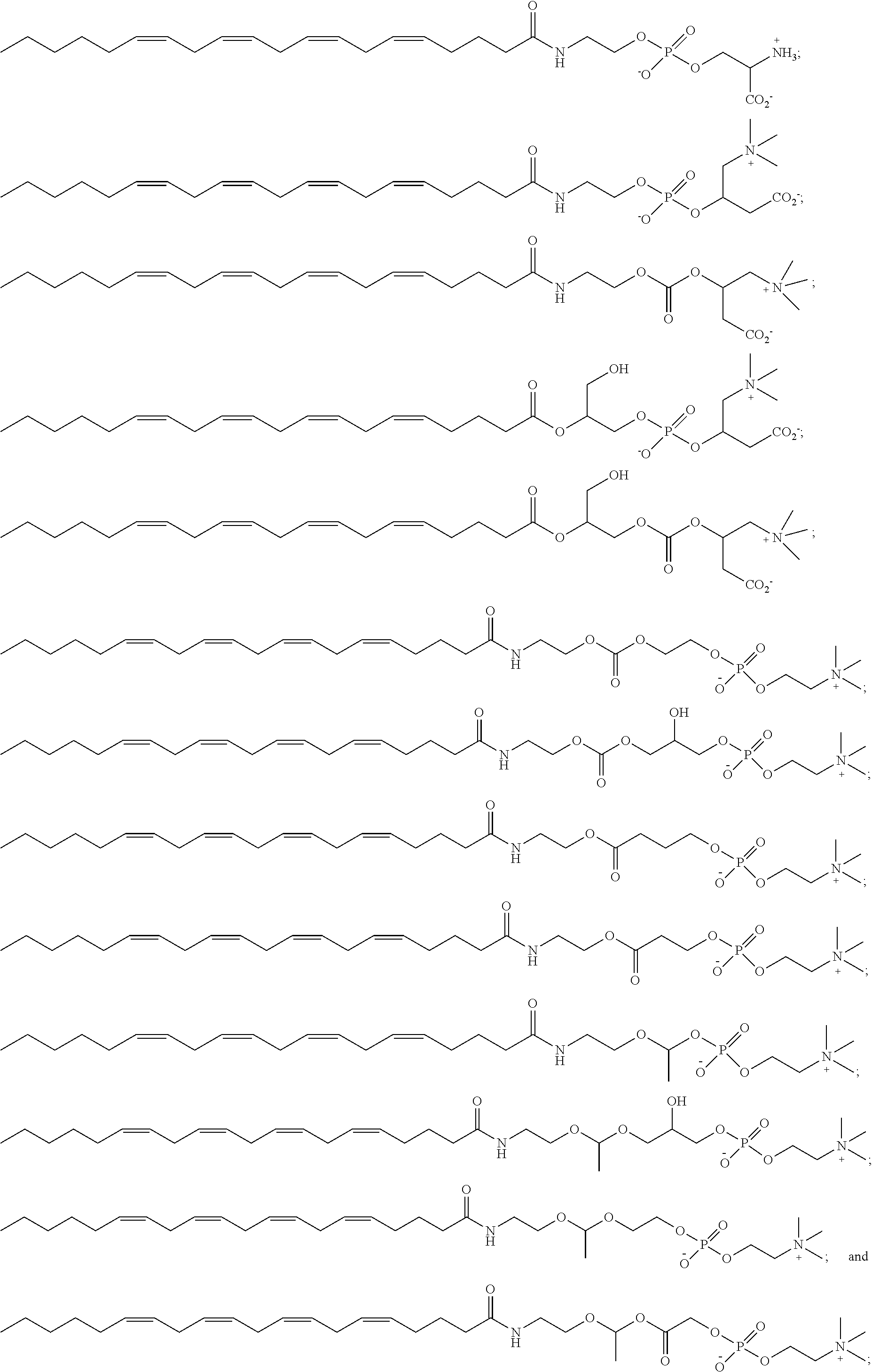

or a pharmaceutically acceptable salt thereof.

In some embodiments, the compound of Formula I or Formula VI is selected from the group consisting of

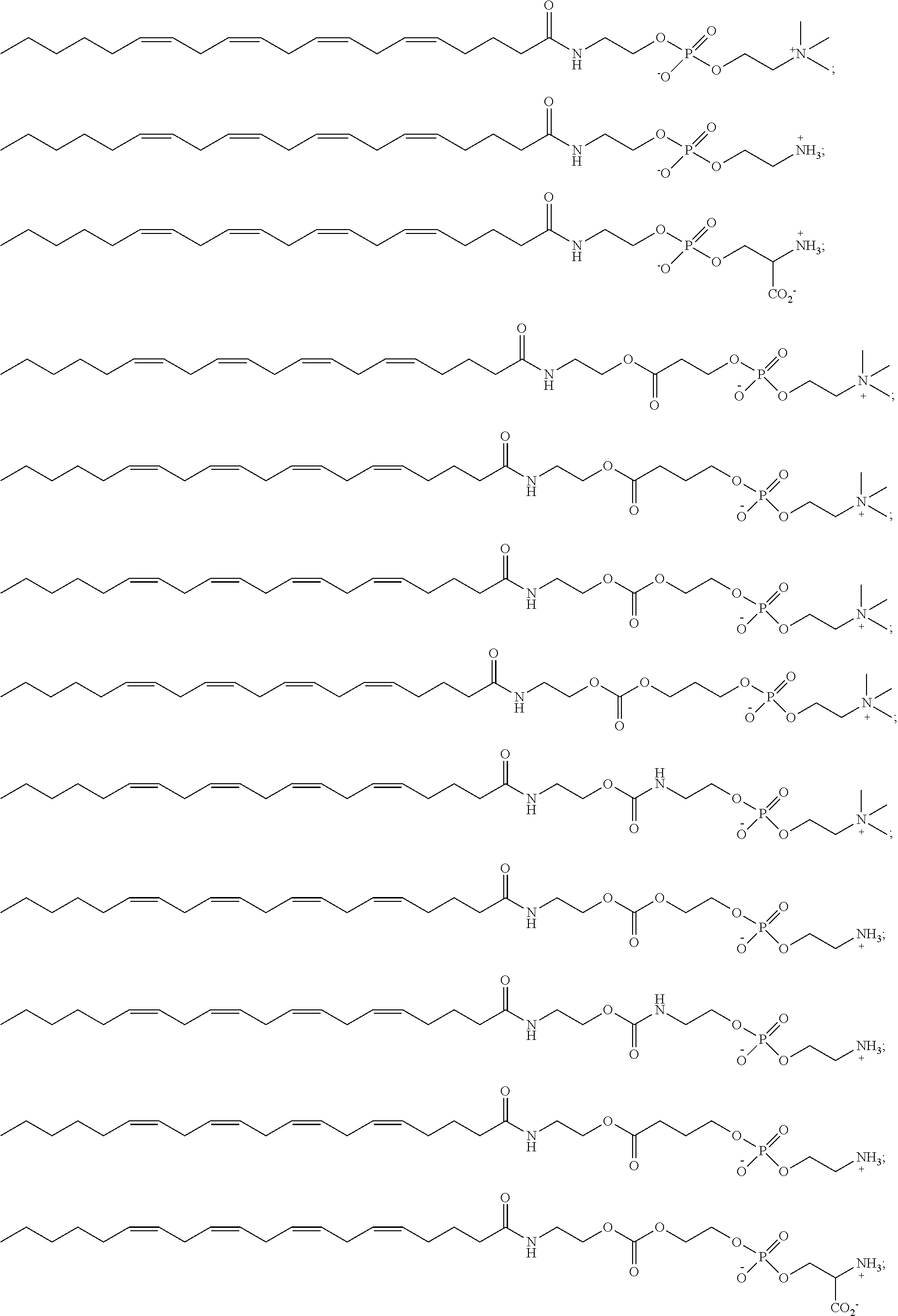

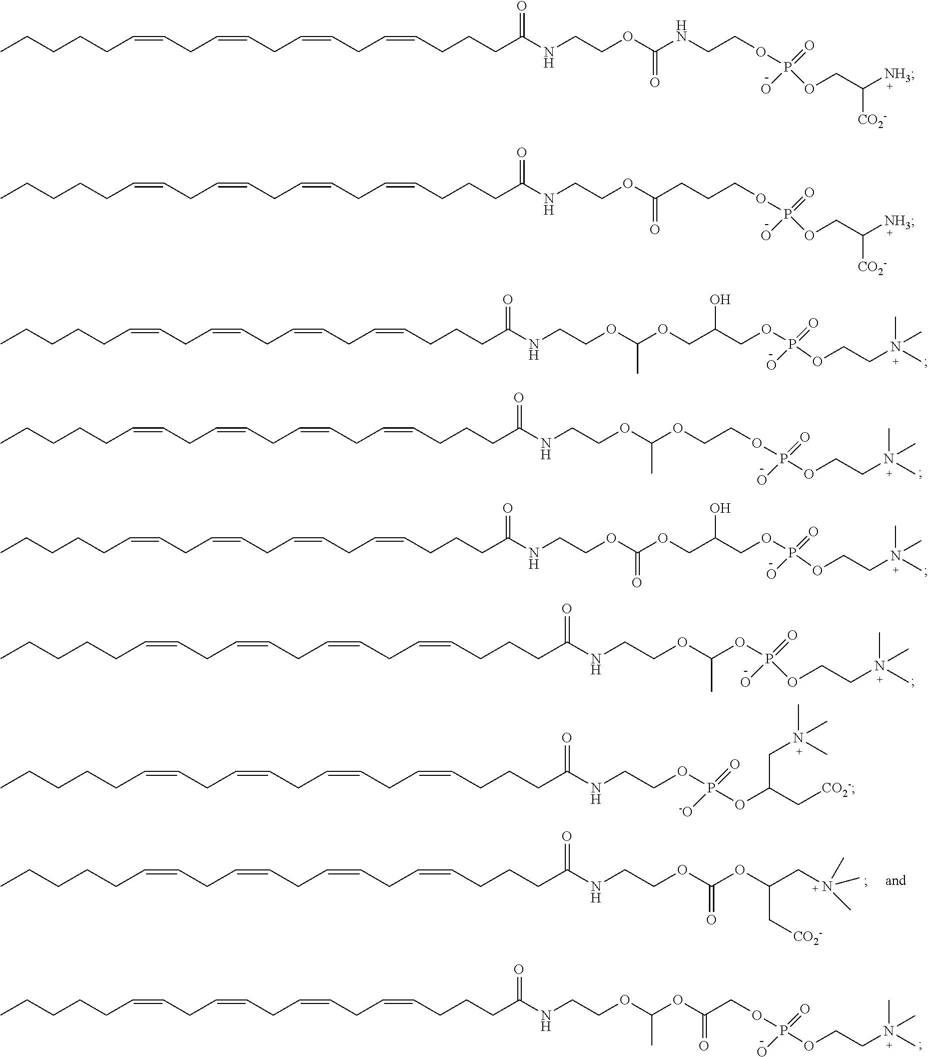

or a pharmaceutically acceptable salt thereof.

In some embodiments, the compound of Formula I or Formula VII is selected from the group consisting of:

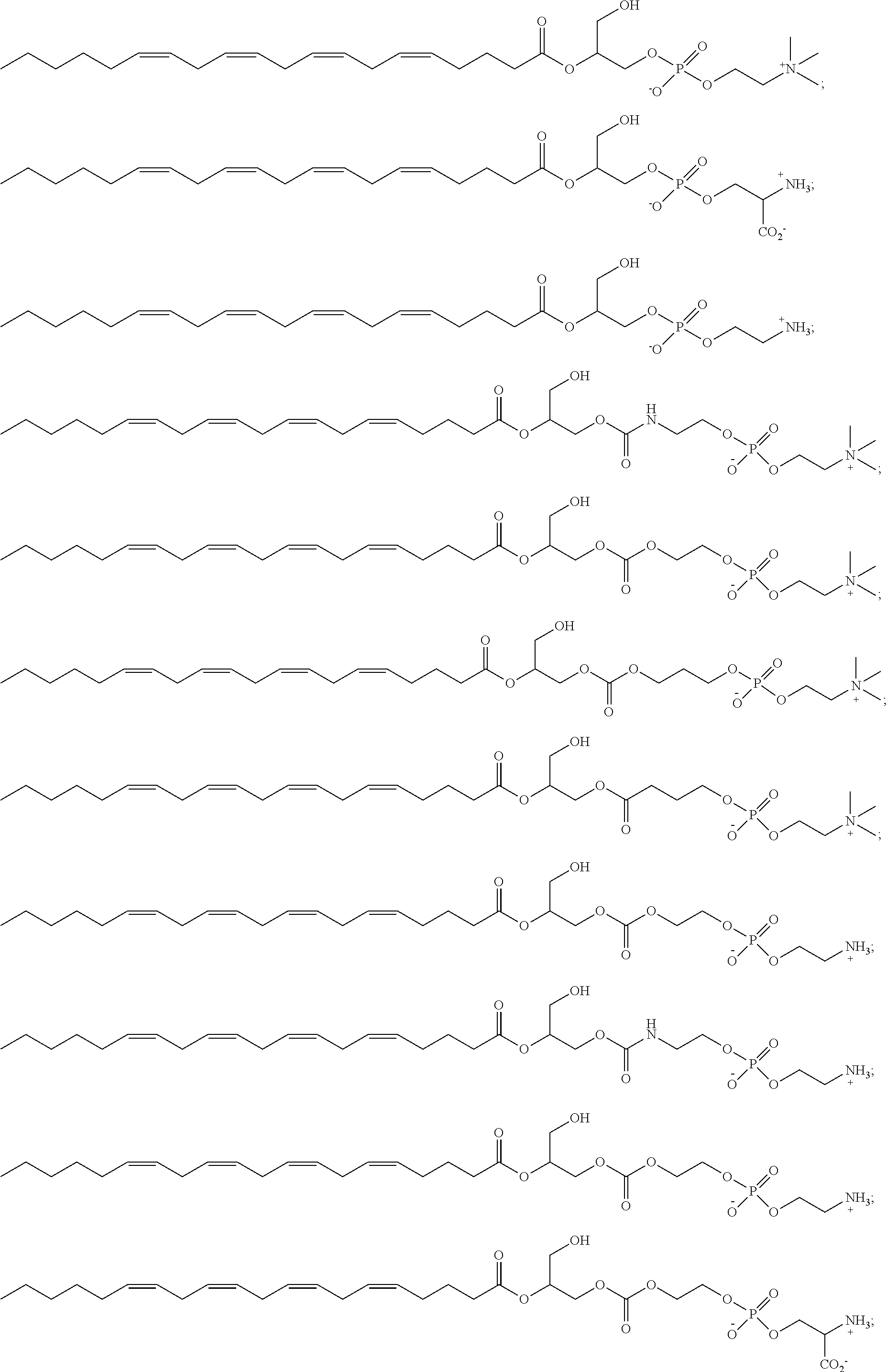

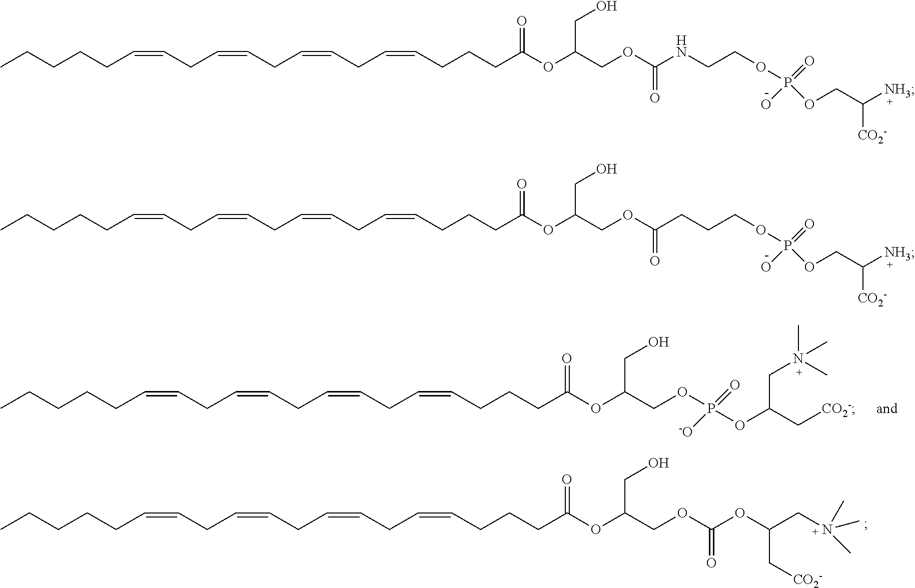

or a pharmaceutically acceptable salt thereof.

In some embodiments, the compound of Formula Ia or Formula VIa is selected from the group consisting of:

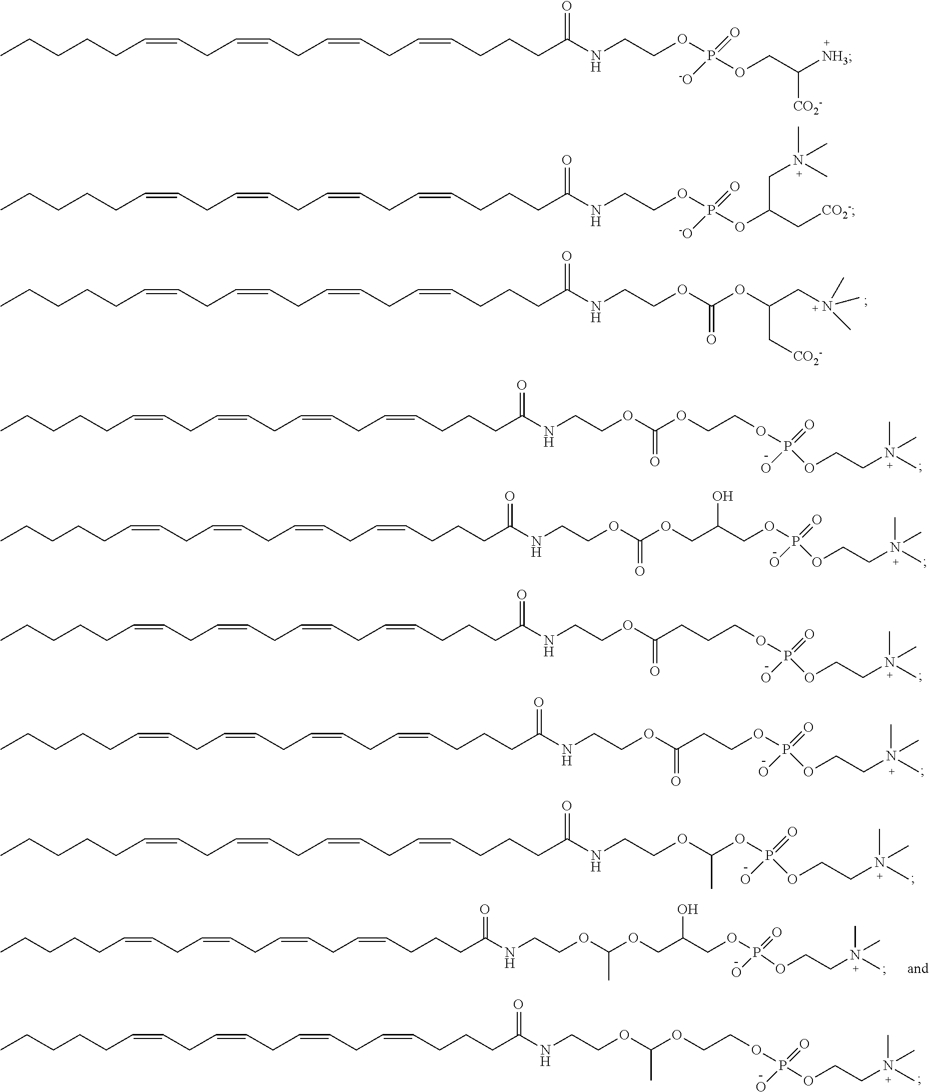

or a pharmaceutically acceptable salt thereof.

In some embodiments, the compound of Formula I or Formula VI is:

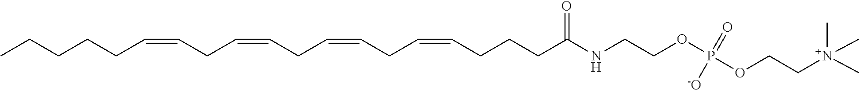

or a pharmaceutically acceptable salt thereof,

### Synthesis

As will be appreciated, the compounds provided herein, including salts thereof, can be prepared using known organic synthesis techniques and can be synthesized according to any of numerous possible synthetic routes.

The compounds provided herein can be prepared, for example, according to the general procedures shown in Schemes 1-2, using appropriately substituted starting materials.

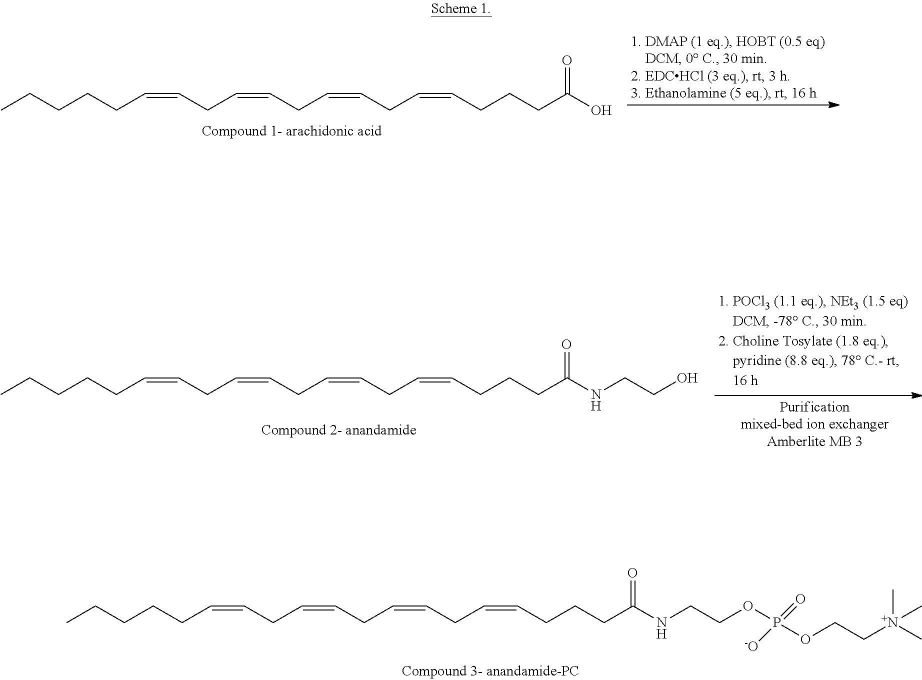

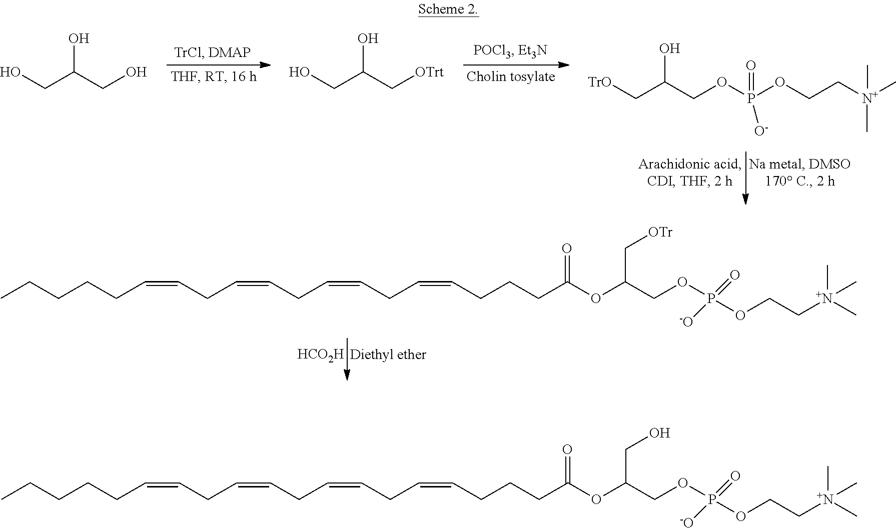

It will be appreciated by one skilled in the art that the processes describe are not the exclusive means by which compounds provided herein may be synthesized and that a broad repertoire of synthetic organic reactions is available to be potentially employed in synthesizing compounds provided herein. The person skilled in the art knows how to select and implement appropriate synthetic routes. Suitable synthetic methods of starting materials, intermediates and products may be identified by reference to the literature, including reference sources such as: Advances in Heterocyclic Chemistry, Vols. 1-107 (Elsevier, 1963-2012); Journal of Heterocyclic Chemistry Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II (Elsevier, 2^{nd} Edition, 2004); Katritzky et al. (Ed.), Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry; Reactions, Mechanisms, and Structure, 6^{th} Ed. (Wiley, 2007); Trost et al. (Ed.), Comprehensive Organic Synthesis (Pergamon Press, 1991).

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wilts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

At various places in the present specification, divalent linking substituents are described. It is specifically intended that each divalent linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups.

As used herein, the phrase “optionally substituted” means unsubstituted or substituted. As used herein, the term “substituted” means that a hydrogen atom is removed and replaced by a substituent. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkylene”, employed alone or in combination with other terms, refers to a divalent alkyl linking group having n to m carbons.

Examples of alkylene groups include, but are not limited to, methylene, ethan-1,2-diyl, propan-1,3-diyl, propan-1,2-diyl, and the like. In some embodiments, the alkylene moiety contains 1 to 6, 1 to 3, or 1 to 2 carbon atoms.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

Example acids can be inorganic or organic acids and include, but are not limited to, strong and weak acids. Some example acids include hydrochloric acid, hydrobromic-acid, sulfuric acid, phosphoric acid, p-toluenesulfonic acid, 4-nitrobenzoic acid, methanesulfonic acid, benzenesulfonic acid, trifluoroacetic acid, and nitric acid. Some weak acids include, but are not limited to acetic acid, propionic acid, butanoic acid, benzoic acid, tartaric acid, pentanoic acid, hexanoic acid, heptanoic acid, octanoic acid, nonanoic acid, and decanoic acid.

Example bases include lithium hydroxide, sodium hydroxide, potassium hydroxide, lithium carbonate, sodium carbonate, potassium carbonate, and sodium bicarbonate. Some example strong bases include, but are not limited to, hydroxide, alkoxides, metal amides, metal hydrides, metal dialkylamides and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include lithium, sodium, and potassium salts of methyl, ethyl, n-propyl, iso-propyl, n-butyl, tert-butyl, trimethylsilyl and cyclohexyl substituted amides.

In some embodiments, the compounds and salts provided herein are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The term, “room temperature” or “RT” as used herein, are understood in the art, and refer generally to a temperature (e.g., a reaction temperature) that is about the temperature of the room in which the reaction is carried out, for example, a temperature from about 20° C. to about 30° C.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Tautomeric forms can be in equilibrium or sterically locked into one form by appropriate substitution.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines, alkali or organic salts of acidic residues such as carboxylic acids; and the like. The pharmaceutically acceptable salts of the present application include the conventional non-toxic salts of the parent compound formed, for example, from non-toxic inorganic or organic acids. The pharmaceutically acceptable salts of the present application can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (MeCN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977). Conventional methods for preparing salt forms are described, for example, in Handbook of Pharmaceutical Salts; Properties, Selection, and Use, Wiley-VCH, 2002.

### Methods of Use

The present application further provides methods of treating a disease or disorder in a subject. As used herein, the term “subject,” refers to any animal, including mammals. Exemplary subjects include, but are not limited to, mice, rats, rabbits, dogs, cats, swine, cattle, sheep, horses, primates, and humans. In some embodiments, the subject is a human. In some embodiments, the method comprises administering to the subject (e.g., a subject in need thereof) a therapeutically effective amount of a compound provided herein (e.g., a compound of Formula I), or a pharmaceutically acceptable salt thereof.

In some embodiments, the disease or disorder is selected from the group consisting of pain, a pain-related disease or disorder, a mood disease or disorder, a disease or disorder of the central nervous system, an optical disease or disorder, cancer, a gastrointestinal disease or disorder, a renal disease or disorder, a renal-related disease or disorder, a cardiovascular disease or disorder, and a skin disease or disorder.

In some embodiments, the disease or disorder is pain or a pain-related disease or disorder. In some embodiments, the pain or a pain-related disease or disorder is selected from the group consisting of acute pain, chronic pain, neuropathic pain, nociceptive pain, inflammatory pain, cancer pain, fibromyalgia, rheumatoid arthritis, osteoarthritis, surgery-related pain, and osteoporosis.

In some embodiments, the disease or disorder is a mood disease or disorder. In some embodiments, the mood disease or disorder is selected from the group consisting of anxiety, depression, a sleeping disorder, an eating disorder, post-traumatic stress disorder, symptoms of drug or alcohol withdrawal, schizophrenia, obsessive-compulsive disorder, bipolar disorder, sexual dysfunction, attention deficit disorder (ADD), and attention deficit hyperactivity disorder (ADHD).

In some embodiments, the disease or disorder is a disease or disorder of the central nervous system or an optical disease or disorder. In some embodiments, the disease or disorder is a disease or disorder of the central nervous system. In some embodiments, the disease or disorder is an optical disease or disorder. In some embodiments, the disease or disorder of the central nervous system or an optical disease or disorder is selected from the group consisting of a demyelinating disease, glaucoma, age-related macular degeneration (AMD), amyotrophic lateral sclerosis (ALS), a cognitive disorder, Alzheimer's disease, a movement disorder, Huntington's chorea, Tourette's syndrome, Niemann-Pick disease, Parkinson's disease, epilepsy, a cerebrovascular disorder, and brain injury.

In some embodiments, the demyelinating disease is selected from the group consisting of multiple sclerosis (MS), neuromyelitis optica (NMO), Devic's disease, central nervous system neuropathy, central pontine myelinolysis, syphilitic myelopathy, leukoencephalopathies, leukodystrophies, Guillain-Barre syndrome, chronic inflammatory demyelinating polyneuropathy, anti-myelin-associated glycoprotein (MAG) peripheral neuropathy, Charcot-Marie-Tooth disease, peripheral neuropathy, myelopathy, optic neuropathy, progressive inflammatory neuropathy, optic neuritis, and transverse myelitis.

In some embodiments, the disease or disorder is cancer. In some embodiments, the cancer is selected from the group consisting of leukemia, mantle cell lymphoma, Hodgkin lymphoma, Non-Hodgkin lymphoma, hepatocellular carcinoma, ovarian cancer, colorectal cancer, pancreatic cancer, prostate cancer, breast cancer, glioma, skin cancer, renal carcinoma and lung cancer

In some embodiments, the disease or disorder is a gastrointestinal disease or disorder. In some embodiments, the gastrointestinal disease or disorder is selected from the group consisting of inflammatory bowel disease, gastroesophageal reflux disease, paralytic ileus, secretory diarrhoea, gastric ulcer, nausea, emesis, and a liver disorder.

In some embodiments, the liver disease is selected from the group consisting of acute liver failure, Alagille syndrome, hepatitis, enlarged liver, Gilbert's syndrome, liver cyst, liver haemangioma, fatty liver disease, steatohepatitis, primary sclerosing cholangitis, fascioliasis, primary bilary cirrhosis, Budd-Chiari syndrome, hemochromatosis, Wilson's disease, and transthyretin-related hereditary amyloidosis.

In some embodiments, the disease or disorder is a renal disease or disorder or a renal-related disease or disorder. In some embodiments, the renal disease or disorder or a renal-related disease or disorder is selected from the group consisting of diabetes, diabetic nephropathy, acute inflammatory kidney injury, renal ischemia urinary incontinence, and overactive bladder.

In some embodiments, the disease or disorder is a skin disease or disorder. In some embodiments, the skin disease or disorder is psoriasis or lupus.

In some embodiments, the disease or disorder is a cardiovascular disease or disorder. In some embodiments, the cardiovascular disease or disorder is selected from the group consisting of cardiovascular disease, vascular inflammation, idiopathic pulmonary fibrosis, and hypertension.

The present application further provides a compound described herein, or a pharmaceutically acceptable salt thereof, for use in any of the methods described herein.

The present application further provides use of a compound described herein, or a pharmaceutically acceptable salt thereof, for the preparation of a medicament for use in any of the methods described herein.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease.

Combination Therapies

One or more additional therapeutic agents such as, for example, chemotherapeutic agents, anesthetics (e.g., for use in combination with a surgical procedure) or other agents useful for treating the diseases or disorders provided herein can be used in combination with the compounds and salts provided herein.

Exemplary anesthetics include, but are not limited to, local anesthetics (e.g., lidocaine, procain, ropivacaine) and general anesthetics (e.g., desflurane, enflurane, halothane, isoflurane, methoxyflurane, nitrous oxide, sevoflurane, mmobarbital, methohexital, thiamylal, thiopental, diazepam, lorazepam, midazolam, etomidate, ketamine, propofol, alfentanil, fentanyl, remifentanil, buprenorphine, butorphanol, hydromorphone levorphanol, meperidine, methadone, morphine, nalbuphine, oxymorphone, and pentazocine).

In some embodiments, the additional therapeutic agent is administered simultaneously with the compound or salt provided herein. In some embodiments, the additional therapeutic agent is administered after administration of the compound or salt provided herein. In some embodiments, the additional therapeutic agent is administered prior to administration of the compound or salt provided herein. In some embodiments, the compound or salt provided herein is administered during a surgical procedure. In some embodiments, the compound or salt provided herein is administered in combination with an additional therapeutic agent during a surgical procedure.

### Pharmaceutical Formulations

When employed as pharmaceuticals, the compounds and salts provided herein can be administered in the form of pharmaceutical compositions. These compositions can be prepared as described herein or elsewhere, and can be administered by a variety of routes, depending upon whether local or systemic treatment is desired and upon the area to be treated. Administration may be topical (e.g., transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial (e.g., intrathecal or intraventricular, administration). Parenteral administration can be in the form of a single bolus dose, or may be, for example, by a continuous perfusion pump. In some embodiments, the compounds provided herein (e.g., compounds of Formula I) are suitable for parenteral administration. In some embodiments, the compounds provided herein (e.g., the compounds of Formula I) are suitable for intravenous administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. In some embodiments, the pharmaceutical compositions provided herein are suitable for parenteral administration. In some embodiments, the compositions provided herein are suitable for intravenous administration.

Also provided are pharmaceutical compositions which contain, as the active ingredient, a compound provided herein, or a pharmaceutically acceptable salt thereof, in combination with one or more pharmaceutically acceptable carriers (excipients). In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

Some examples of suitable excipients include, without limitation, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include, without limitation, lubricating agents such as talc, magnesium stearate, and mineral oil, wetting agents; emulsifying and suspending agents; preserving agents such as methyl- and propylhydroxy-benzoates; sweetening agents; flavoring agents, or combinations thereof.

### EXAMPLES

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results. Analytical methods described throughout the Examples were performed according to the following procedures:

LC-MS - Method A

| | |
|---|---|
| Instrument | Shimadzu |
| Injection volume | 3 μL |
| Solvent A | ACN |
| Solvent B | Water + 0.01% HCOOH |
| Flow rate | 0.5 ml/min |
| Temperature | Ambient |
| Column | LUNA-C18, 50 × 2.1 mm, 1.6 μm |

Solvent Gradient

| Time (min) | Solvent B[%] |
|---|---|
| 0 | 95 |
| 8 | 10 |
| 10 | 10 |

LC-MS - Method B

| | |
|---|---|
| Instrument | Shimadzu |
| Injection volume | 3 μL |
| Solvent A | ACN |
| Solvent B | Water + 5 mM NH₄HCO₃ |
| Flow rate | 0.5 ml/min |
| Temperature | Ambient |
| Column | LUNA-C18, 50 × 2.1 mm, 1.6 μm |

Solvent Gradient

| Time (min) | Solvent B[%] |
|---|---|
| 0 | 95 |
| 8 | 10 |
| 10 | 10 |

LC-MS - Method C

| | |
|---|---|
| Instrument | Shimadzu |
| Injection volume | 8 μL |
| Solvent A | Water + 0.1% HCOOH |
| Solvent B | ACN |
| Flow rate | 0.5 mL/min |
| Temperature | Ambient |
| Column | KINETEX EVO, 50 × 2.1 mm, 1.6 μm |

Solvent Gradient

| Time (min) | Solvent B[%] |
|---|---|
| 0 | 5 |
| 0.5 | 5 |
| 7 | 95 |
| 12 | 95 |

LC-MS - Method D

| | |
|---|---|
| Instrument | Agilent |
| Injection volume | 3/8 μL |
| Solvent A | Water + 0.1% HCOOH |
| Solvent B | ACN |
| Flow rate | 0.5 mL/min |
| Temperature | Ambient |
| Column | LUNA OMEGA POLAR (50 × 2.1 mm), 1.6 μm |

Solvent Gradient

| Time (min) | Solvent B[%] |
|---|---|
| 0.01 | 5 |
| 1.8 | 95 |
| 4.5 | 95 |

LC-MS - Method E

| | |
|---|---|
| Instrument | Agilent |
| Injection volume | 3 μL |
| Solvent A | Water + 0.1% HCOOH |
| Solvent B | ACN |
| Flow rate | 0.5 mL/min |
| Temperature | Ambient |
| Column | Kinetex, EVO, C18, 50 × 2.1 mm, 1.6 μm |

Solvent Gradient

| Time (min) | Solvent B[%] |
|---|---|
| 0.01 | 5 |
| 1.8 | 95 |
| 8.5 | 95 |

LC-MS - Method F

| | |
|---|---|
| Instrument | Shimadzu |
| Injection volume | 3 μL |
| Solvent A | Water + 0.01% HCOOH |
| Solvent B | CAN |
| Flow rate | 0.6 ml/min |
| Temperature | Ambient |
| Column | Gemini C18, 50 × 2.0 mm, 3.0 μm |

Solvent Gradient

| Time (min) | Solvent B[%] |
|---|---|
| 0 | 5 |
| 8 | 90 |
| 12 | 90 |

PREP-HPLC Method A

| | |
|---|---|
| Instrument | Shimadzu |
| Injection volume | 80 mg per injection |
| Solvent A | ACN |
| Solvent B | Water |
| Flow rate | 30 mL/min |
| Temperature | Ambient |
| Column | LUNA-C18, 250 × 21.2 mm, 1.6 μm |

Solvent Gradient

| Time (min) | Solvent B[%] |
|---|---|
| 0 | 80 |
| 10 | 50 |
| 22 | 50 |
| 35 | 90 |

PREP-HPLC Method B

| | |
|---|---|
| Instrument | Agilent |
| Injection volume | 10 mg per injection |
| Solvent A | ACN |
| Solvent B | 0.1% of Formic acid in Water |
| Flow rate | 15 ml/min |
| Temperature | Ambient |
| Column | LUNA-C18, 250 × 21.2 mm, 5 μm |

Solvent Gradient

| Time (min) | Solvent B[%] |
|---|---|
| 0 | 10 |
| 15 | 80 |
| 30 | 95 |

PREP-HPLC Method C

| | |
|---|---|
| Instrument | Agilent |
| Injection volume | 20 mg per injection |
| Solvent A | ACN |
| Solvent B | 0.1% of Formic acid in Water |
| Flow rate | 15 ml/min |
| Temperature | Ambient |
| Column | Gemini C18, 250 × 21.2 mm, 5 μm |

Solvent Gradient

| Time (min) | Solvent B[%] |
|---|---|
| 0 | 10 |
| 15 | 70 |
| 30 | 95 |

PREP-HPLC Method D

| | |
|---|---|
| Instrument | Shimadzu |
| Injection volume | 80 mg per injection |
| Solvent A | ACN |
| Solvent B | 0.1% of Formic acid in Water |
| Flow rate | 15 ml/min |
| Temperature | Ambient |
| Column | Gemini-C18, 250 × 21.2 mm, 5.0 μm |

Solvent Gradient

| Time (min) | Solvent B[%] |
|---|---|
| 0 | 30 |
| 20 | 75 |
| 25 | 95 |

PREP-HPLC Method E

| | |
|---|---|
| Instrument | Agilent |
| Injection volume | 50 mg per injection |
| Solvent A | ACN |
| Solvent B | 0.1% of Formic acid in Water |
| Flow rate | 15 ml/min |
| Temperature | Ambient |
| Column | Gemini-C18, 250 × 21.2 mm, 5.0 μm |

Solvent Gradient

| Time (min) | Solvent B[%] |
|---|---|
| 0 | 70 |
| 20 | 20 |

PREP-HPLC Method F

| | |
|---|---|
| Instrument | Agilent |
| Injection volume | 50 mg per injection |
| Solvent A | ACN |
| Solvent B | 0.1% of Formic acid in Water |
| Flow rate | 15 ml/min |
| Temperature | Ambient |
| Column | Gemini-C18, 250 × 21.2 mm, 5.0 μm |

Solvent Gradient

| Time (min) | Solvent B[%] |
|---|---|
| 0 | 60 |
| 20 | 30 |
| 25 | 5 |

MS/MS TUNE PARAMETERS

| | |
|---|---|
| Instrument | AB Sciex QTRAP 5500 |
| Column | Cortecs UPLC C18 30 mm × 2.1 mm × 1.6 μM |
| Solvent A | Water + 0.1% Formic Acid |
| Solvent B | Acetonitrile + 0.1% Formic Acid |

| Time (min) | Flow rate (mL/min) | % A | % B |
|---|---|---|---|
| 0.1 | 0.650 | 95 | 5 |
| 0.7 | 0.650 | 5 | 95 |
| 0.9 | 0.650 | 5 | 95 |
| 1.0 | 0.650 | 95 | 5 |
| 1.1 | 0.650 | 95 | 5 |

ADDITIONAL MS/MS PARAMETERS

| Parameter | |
|---|---|
| Curtain gas | 35 |
| Collision gas | medium/low |
| Ionspray (V) | 5500 |
| Temperature | 650/600 |
| Ion source gas 1 | 30/50 |
| Ion source gas 2 | 60/50 |

### Intermediate 1. 2-hydroxyethyl (2-(trimethylammonio)ethyl) phosphate

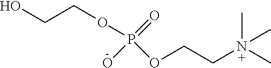

### Step 1. 2-(benzyloxy)ethan-1-ol

To a stirred solution of NaH (1.93 g, 80,645 mmol, 1.0 eq.) in THF (50 mL) at 0° C. was added Ethylene glycol (5 g, 80.645 mmol, 1.0 eq.), followed by catalytic amount of TBAI (100 mg). The reaction mixture was stirred for 0.10 min at 0° C. and then stirred at RT for 1 h. Benzyl bromide (13.7 mL, 80.645 mmol, 1.0 eq.) was then added drop wise to the reaction mixture at 0° C. Then the reaction was left to stir at 25° C. The reaction mixture was quenched with cold water (250 mL) and extracted with EtOAc (3×250 mL). The combined organic layer was dried over anhydrous Na₂SO₄ and concentrated under vacuum. The obtained crude product (9 g) was purified via silica gel chromatography using gradient elution with 30% EtOAc/Hexane, giving the title compound as a pale-yellow oil (2.5 g). ¹H NMR (CDCl₃): δ 7.38-7.30 (m, 5H), 4.56 (s, 2H), 3.78-3.74 (m, 2H), 3.61-3.59 (m, 2H), 2.06 (t, J=6.4 Hz, 1H).

### Step 2. 2-(benzyloxy)ethyl (2-(trimethylammonio)ethyl) phosphate

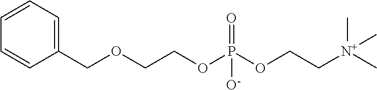 [SMILES: C[N+](C)(C)CCOP(=O)([O-])OCCOCc1ccccc1]

To a stirred solution of 2-(benzyloxy)ethan-1-ol (1 g, 6.535 mmol, 1 eq.) in chloroform (10 mL) was added Et₃N (1.18 mL, 8.169 mmol, 1.25 eq.) followed by POCl₃ (0.78 mL, 8.169 mmol, 1.25 eq.) at −10° C. Then the reaction mixture was stirred for 1 h at 25° C. After 1 h, was added pyridine (2.5 mL, 56.208 mmol, 8.6 eq.) followed by choline tosylate (2.6 g, 9.803 mmol, 1.5 eq) at −10° C. and the reaction with as stirred at 25° C. for 18 h. The reaction mass was then cooled to 0° C. and quenched with water (5 mL) and extracted with DCM (3×50 mL). The aqueous layer was purified from flash chromatography (Method: Column: Biotage-C18, 60 g Duo-100 Å 30 μm; Mobile phase: (ACN:Water+0.1% TFA); B %:B %: 0-8%, 0-20 min/8% 20-45 min), pure fractions were lyophilized to afford the title compound as a colorless liquid (1.1 g). [M+H]⁺ (m/z): 318.4; ¹H NMR (CDCl₃): δ 7.34-7.28 (m, 5H), 4.46 (s, 2H), 4.25 (bs, 2H), 4.04 (bs, 2H), 3.64 (bs, 2H), 3.52 (bs, 2H), 3.03 (s, 9H). ³¹P NMR: δ −2.23.

### Step 3. 2-hydroxyethyl (2-(trimethylammonio)ethyl) phosphate

To a stirred solution of 2-(benzyloxy)ethyl (2-(trimethylammonio)ethyl) phosphate (300 mg, 0.946 mmol, 1.0 eq.) in IP A (3 ml) was added 10% Pd/C (50% wet) (50 mg) at 25° C. Then the reaction mixture was stirred under hydrogen pressure at 25° C. for 18 h. The reaction mixture was filtered through celite pad, washed with IP A, and the resulting filtrate was concentrated to dryness. The obtained crude residue was dried under vacuum to afford the title compound as a colourless liquid (200 mg). [M+H]⁺ (m/z): 227.5; ¹H NMR. (CD₃OD): 4.32-4.28 (m, 2H), 3.96-3.92 (m, 2H), 3.70 (t, J=4.8 Hz, 2H), 3.65-3.63 (m, 2H), 3.22 (s, 9H). ³¹P NMR: δ −0.17.

### Intermediate 2. 3-hydroxypropyl (2-(trimethylammonio)ethyl) phosphate

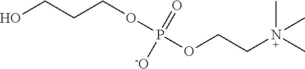

### Step 1. 3-(benzyloxy)propan-1-ol

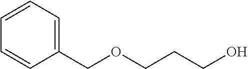 [SMILES: OCCCOCc1ccccc1]

To a stirred solution of NaH (1.5 g, 65.703 mmol, 1.0 eq.) in THF (50 mL) at 0° C. was added propane-1,3-diol (5 g, 65.703 mol, 1.0 eq.), followed by catalytic amount of tetrabutylammonium iodide (TBAI; 100 mg). Then the reaction mixture was stirred for 10 min at 0° C. and then stirred at RT for 1 h. Benzyl bromide (7.8 mL, 65.703 mol, 1.0 eq.) was then added to the reaction mixture drop wise at 0° C. The reaction mixture was stirred for 18 h at 25° C. and was then quenched with ice water (250 mL) and extracted with EtOAc (3×250 mL). The combined organic layers were dried over anhydrous Na₂SO₄ and concentrated to dryness. The obtained crude residue (11 g) was purified via silica gel chromatography using gradient elution with 30% EtOAc/Hexane to afford the title compound as a pale-yellow liquid (4.6 g). ¹H NMR (CDCl₃): δ 7.37-7.27 (m, 5H), 4.52 (s, 2H), 3.79 (q, J=11.2, 5.6 Hz, 2H), 3.66 (t, J=6 Hz, 2H), 2.26 (t, J=5.6 Hz, 1H), 1.90-1.84 (m, 2H).

### Step 2. 3˜(benzyloxy)propyl (2-(trimethylammonio)ethyl) phosphate

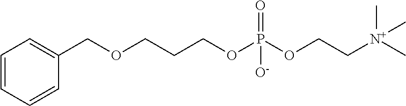 [SMILES: C[N+](C)(C)CCOP(=O)([O-])OCCCOCc1ccccc1]

To a stirred solution of 3-(benzyloxy)propan-1-ol (1 g, 5.988 mmol, 1 eq.) in chloroform (10 mL) was added Et₃N (1.05 mL, 7,485 mmol, 1.25 eq.) and followed by POCl₃ (0.699 mL, 7.485 mmol, 1.25 eq.) at −10° C. The reaction mixture was stirred at 25° C. for 1 h. Pyridine (2.5 mL, 51.496 mmol, 8.6 eq.) and choline tosylate (2.47 g, 8.982 mmol, 1.5 eq) at −10° C. were added to the reaction mixture and the reaction was stirred at 25° C. After 18 h, the reaction mixture was cooled to 0° C., water (5 mL) was added, and the mixture was extracted with DCM (3×20 mL). The aqueous layer was purified via flash chromatography (Method: Column: Biotage-C18, 60 g Duo-100 Å 30 μm; Mobile phase: (ACN:Water+0.1% TFA]; B %:B %: 0-8%, 0-20 min/8% 20-45 min). Pure fractions were lyophilized to afford the title compound as a colorless liquid (430 mg). [M+H]⁺ (m/z): 332.4; ¹H NMR (CDCl₃): δ 7.36-7.25 (m, 5H), 5.55 (bs, 4H), 4.44 (s, 2H), 4.28 (bs, 2H), 4.01 (q, J=12.8, 6.4 Hz, 2H), 3.60 (bs, 2H), 3.57 (t, J=6 Hz, 2H), 3.09 (s, 9H), 1.94-1.88 (m, 2H). ³¹P NMR: δ −2.28.

### Step 3. 3-hydroxypropyl (2-(trimethylammonio)ethyl) phosphate

To a stirred solution of 3-(benzyloxy)propyl (2-(trimethylammonio)ethyl) phosphate (100 mg, 0.302 mmol, 1.0 eq.) in IPA (1 ml) was added 10% Pd/C (50% wet) (10 mg) at 25° C. The reaction mixture was stirred under hydrogen pressure at 25° C. for 18 h, filtered through celite pad, and washed with IPA. The filtrate was then concentrated and the obtained crude residue was dried under reduced pressure to afford the title compound (60 mg) as a colorless liquid (60 mg). [M+Na]⁺ (m/z): 264.1; ¹H NMR (CD₃OD): 4.26 (bs, 2H), 3.98 (q, J=12.4, 6 Hz, 2H), 3.67 (t, J=6 Hz, 2H), 3.64-3.61 (m, 2H), 3.22 (s, 9H), 1.86-1.79 (m, 2H). ³¹P NMR: δ −0.03.

### Intermediate 3. 2-aminoethyl (2-(trimethylammonio)ethyl) phosphate

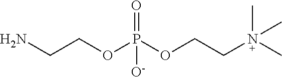

### Step 1. benzyl (2-hydroxyethyl)carbamate

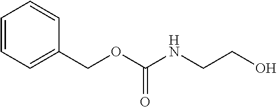 [SMILES: O=C(NCCO)OCc1ccccc1]

To a stirred solution of 2-aminoethan-1-ol (50 g, 0.819 mol, 1.0 eq.) in DCM (1.5 L) at 0° C. was added Et₃N (137 mL, 0,983 mol, 1.2 eq.) dropwise. The reaction mixture was stirred for 10 min at 0° C. and after 10 min, benzyl carbonochloridate (Cbz-Cl; 50%; 302 mL, 1.064 mol, 1.2 eq) was added to the reaction mixture, drop wise at 0° C. Then the reaction was stirred for 3 h at 0° C. and monitored by thin layer chromatography (TLC). The reaction mixture was then quenched with water (500 mL) and extracted with DCM (3×500 mL). The total organic layer was then dried over anhydrous Na₂SO₄, filtered, and concentrated to obtain crude (200 g) compound, winch was purified by silica gel column chromatography using gradient elution with 3% MeOH/DCM to afford benzyl (2-hydroxyethyl)carbamate (95 g) as a white solid. Mass [m/z]: 196.09 [M+H]⁺. Yield: 95 g (59.7%).

### Step 2. 2-(((benzyloxy)carbonyl)amino)ethyl(2-trimethylammonio)ethyl)phosphate

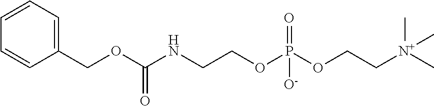 [SMILES: C[N+](C)(C)CCOP(=O)([O-])OCCNC(=O)OCc1ccccc1]

To a stirred solution of benzyl (2-hydroxyethyl)carbamate (5 g, 25.641 mmol, 1 eq.) in chloroform (100 mL) was added Et₃N (5.5 mL, 38.461 mmol, 1.5 eq.) followed by POCl₃ (2.65 mL, 28.205 mmol, 1.1 eq.) at −10° C. Then the reaction mixture was stirred for 1 h at 25° C., and monitored by TLC. After 1 h, pyridine (17.5 mL, 220.512 mmol, 8.6 eq.) and choline tosylate (10.55 g, 38.461 mmol, 1.5 eq) were added at −10° C. and the resulting mixture was stirred at 25° C. for 18 h. The reaction mass was then cooled to 0° C., quenched with water (20 mL), and extracted with DCM (3×100 mL). The aqueous layer was purified by flash chromatography [Column: Biotage-C18, 60 g Duo-100 Å 30 μm; Mobile phase: [ACN:Water+0.1% TFA]; B %:B %: 0-8%, 0-20 min/8% 20-45 min,] to afford 2-(((benzyloxy)carbonyl)amino)ethyl(2-(trimethylammonio)ethyl)phosphate (2.4 g) as a colorless liquid. Mass [m/z]: 361.01 [M+H]⁺. Yield: 2.4 g (26%).

### Step 3. 2-aminoethyl(2-(trimethylammonio)ethyl)phosphate

To a stirred solution of 2-(((benzyloxy)carbonyl)amino)ethyl(2-(trimethylammonio)ethyl)phosphate (2.3 g, 0.638 mmol, 1.0 eq.) in isopropyl alcohol (IPA; 20 mL) was added 10% Pd/C (50% wet; 500 mg) at 25° C. The reaction mixture was stirred under hydrogen pressure at 25° C. for 18 h. The resulting mixture was filtered through a celite pad and washed with IPA. The filtrate was concentrated and dried under vacuum to yield 2-aminoethyl(2-(trimethylammonio)ethyl)phosphate (1.6 g) as a colorless liquid. The product was used in the following Examples without further purification. Mass [m/z]: 227.5 [M+H]⁺. Yield: 1.6 g (93%).

### Example 1. (5Z,8Z,11Z,14Z)—N-(2-hydroxyethyl)icosa-5,8,11,14-tetraenamide (Compound 2; Anandamide)

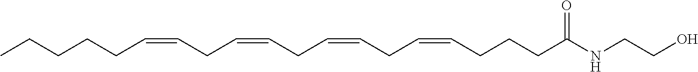 [SMILES: CCCCC/C=C\C/C=C\C/C=C\C/C=C\CCCC(=O)NCCO]

To a stirred solution of arachidonic acid (Compound 1) (5 g, 1.64 mmol, 1.0 eq.) in CH₂Cl₂ (100 mL) was added DMAP (2.0 g, 1.64 mmol, 1.0 eq.) and HOBT (1.1 g, 0.82 mmol, 0.5 eq.) in portion wise at room temperature. The reaction mixture was stirred at 0° C. After being stirred for 30 min, EDCl.HCl (9.45 g, 4.93 mmol, 3.0 eq.) was added to the reaction mixture at 0° C.; the mixture was then warmed to room temperature and continued stirring for 3 h. Next, ethanol amine (5.0 mL, 8.22 mmol, 5.0 eq.) was added dropwise and the reaction mixture was stirred for 16 h at room temperature. The reaction mixture was quenched with water (200 mL) and extracted with DCM (2×300 mL). The combined organic layer was washed with 2N HCl (150 mL) followed by sodium chloride solution (150 mL), dried over anhydrous sodium sulfate, filtered, and concentrated under reduced pressure. The obtained residue was further purified by silica-gel column chromatography using ethyl acetate:hexane (15:85) as an eluent to afford anandamide (Compound 2) (3.8 g) as a light yellow colored liquid. [M+H]⁺ (m/z): 348.2; ¹H-NMR (400 MHz, CDCl₃): δ 5.94 (bs, 0.8H), 5.44-5.30 (m, 8H), 3.73-3.71 (m, 2H), 3.44-3.35 (m, 2H), 2.85-2.79 (m, 6H), 2.22 (t, J=7.6 Hz, 2H), 2.10-2.03 (m, 4H), 1.77-1.69 (m, 3H), 1.39-1.24 (m, 7H), 0.88 (t, J=6.8 Hz, 3H).

### Example 2. 2-(5Z,8Z,11Z,14Z)-icosa-5,8,11,14-tetraenamidoethyl (trimethylammonio) ethylphosphate (Compound 3; Anandamide-PC)

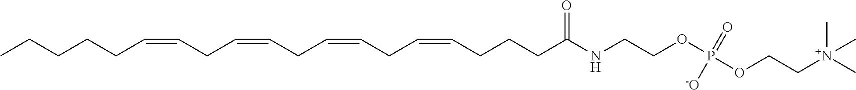

To a stirred solution of anandamide (Compound 2) (2.5 g, 7.20 mmol, 1.0 eq.) in anhydrous CH₂Cl₂ (30 mL) was added triethyl amine (1.5 mL, 10.8 mmol, 1.5 eq.), followed by dropwise addition of POCl₃ (0.72 mL, 7.92 mmol, 0.1.1 eq. in 1 ml, of CH₂Cl₂) and the resulting mixture was stirred for 30 min at −78° C. Dry pyridine (4.9 mL, 61.9 mmol, 8.6 eq.) and choline tosylate (2.85 g, 10.8 mmol, 1.5 eq.) were added at −78° C. and the mixture was stirred at room temperature for 16 h. The reaction mixture was cooled to 0° C., quenched with water (20 mL) and stirred for 1 h, then extracted with 10% MeOH/DCM (2×50 mL), The combined organic layer was dried over anhydrous sodium sulfate and filtered. The solvent was concentrated under reduced pressure to afford a crude residue, which was dissolved in DCM (50 mL) and washed with MeOH:H₂O (1:1, 32 mL) followed by 3% Na₂CO₃/MeOH (1:1, 20 mL) and further with MeOH:H₂O (1:9, 30 mL). The combined organic layer was dried over sodium sulfate, filtered, concentrated under reduced pressure, and co-distilled with isopropyl alcohol (2 mL) to dryness. The resulting residue was triturated in acetone (55 mL) for about 30 min, filtered, and concentrated under reduced pressure at 25-30° C. to obtain partially pure material, which was dissolved in EtOH (55 mL) and filtered to remove insoluble particulates. The filtrate was treated with 30 ml, of Amberlite MB 3® resin. After stirring for 4 h, the solvent was filtered and concentrated to afford crude product which was purified by PREP-HPLC (Column: LUNA-C18, 250×21.2 mm, 1.6 μm; Mobile phase: [CH₃CN:Water]; B %: 80%-50%, 22 min.; 50%-90% 35 min) to afford anandamide-PC (Compound 3) (170 mg) as a light brown color solid. [M+H]⁺ (m/z): 513.2, ¹H-NMR (400 MHz, DMSO-d₆): δ 8.35 (bs, 1H), 5.34-5.28 (m, 8H), 4.03-4.02 (m, 2H), 3.67-3.62 (m, 2H), 3.52-3.49 (t, J=4.8 Hz, 2H). 3.16-3.12 (m, 11H), 2.82-2.77 (m, 6H), 2.08-2.00 (m, 6H), 1.59-1.50 (m, 2H), 1.32-1.24 (m, 6H), 0.860 (t, J=6.8 Hz, 3H). ¹³C-NMR (100 MHz, DMSO-d₆): δ 172.27, 130.42, 129.92, 128.59, 128.50, 128.46, 128.26, 128.16, 128.00, 65.94, 63.07, 63.01, 58.77, 58.72, 53.57, 35.38, 31.35, 29.18, 27.08, 26.81, 25.72, 25.67, 22.44, 14.40. ³¹P-NMR (161.9 MHz, DMSO-d₆): δ −0.27.

### Example 3. 2-ammonioethyl (2-((5Z,8Z,11Z,14Z)-icosa-5,8,11,14-tetraenamido)ethyl) phosphate (Compound 4)

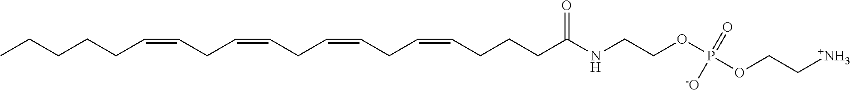

### Step-1. 2-(((benzyloxy) carbonyl)amino) ethyl(2((tertbutoxycarbonyl) amino)ethyl) phosphate

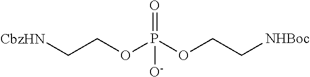 [SMILES: CC(C)(C)OC(=O)NCCOP(=O)([O-])OCCNC(=O)OCc1ccccc1]

To a stirred solution of benzyl (2-hydroxyethyl)carbamate (1 g, 5,128 mmol, 1 eq.) in chloroform (20 mL) was added Et₃N (1.1 mL, 7.692 mmol, 1.5 eq.) followed by POCl₃ (0.52 mL, 5.641 mmol, 1.1 eq.) at −10° C. The reaction mixture was then stirred for 1 h at 25° C., followed by the addition of pyridine (3.6 mL, 44.102 mmol, 8.6 eq.) and N-Boc ethanol amine (1.23 g, 7.692 mmol, 1.5 eq) at −10° C. The reaction mixture was stirred for 18 h at 25° C. The mixture was then cooled to 0° C., water was added (20 mL), and the mixture was extracted with dichloromethane (DCM; 3×30 mL). The organic layer was concentrated and purified by flash chromatography Column: Biotage-C18, 30 g Duo-100 Å 30 μm; Mobile phase: ACN:Water+0.015% NH₄HCO₃; min/B %: 0/0, 5/5, 30/5, 35/14, 55/14, 58/100. The pure fractions were lyophilized to afford the title compound (200 mg) as off-white solid. [M−H]⁺ (m/z): 417.2; ¹H-NMR (400 MHz, DMSO-d₆): δ 7.69 (br s, 1H), 7.38-7.27 (m, 5H), 7.09 (br s, 1H), 4.99 (s, 2H), 3.72-3.57 (m, 4H). 3.14-3.10 (m, 2H), 3.05-3.01 (m, 2H), 1.36 (9H), ³¹P NMR (400 MHz, DMSO-d₆): 0.14.

### Step 2. 2-aminoethyl (2-((tert-butoxycarbonyl)amino)ethyl) phosphate

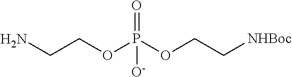 [SMILES: CC(C)(C)OC(=O)NCCOP(=O)([O-])OCCN]

To a stirred solution of 2(((benzyloxy) carbonyl)amino) ethyl(2((tertbutoxycarbonyl) amino)ethyl) phosphate (50 mg, 0.119 mmol, 1.0 eq.) in isopropanol (IPA; 2 mL) was added 10% Pd/C (50% wet) (15 mg) at 25° C. The reaction mixture was stirred under hydrogen atmosphere at 25° C. for 2 h and was filtered through celite pad, and washed with IP A (5 mL). The filtrate was concentrated and dried under vacuum to afford the title product (40 mg, crude) as off-white solid, which was used for the next step without purification. [M+H]⁺ (m/z): 285.2; ¹H-NMR (400 MHz, DMSO-d₆): δ 8.36 (br s, 2H), 6.96 (br s, 1H), 3.87-3.79 (m, 2H), 3.68-3.60 (m, 2H). 3.10-3.02 (m, 2H), 2.97-2.92 (m, 2H), 1.37 (s, 9H). ³¹P NMR (400 MHz, DMSO-d₆): 0.82.

### Step 3. 2-((tert-butoxycarbonyl)amino)ethyl (2-((5Z,8Z,11Z,14Z)-icosa-5,8,11,14-tetraenamido)ethyl) phosphate

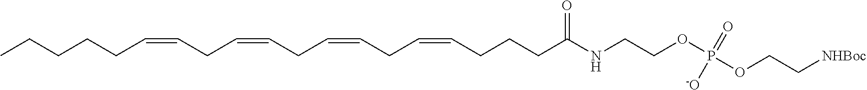

To a stirred solution of arachidonic acid (43 mg, 0.141 mmol, 1.0 eq.) in DMF (2 mL) was added HATU (64 mg, 0.167 mmol, 1.2 eq.) and stirred for 30 min. After 30 min, DIPEA (0.07 mL, 0.424 mmol, 3.0 eq.) and 2-aminoethyl (2-((tert-butoxycarbonyl)amino)ethyl) phosphate (40 mg, 0.141 mmol, 1.0 eq.) were added to the reaction mixture at 25° C. The mixture was stirred at room temperature for 18 h.

The solvent was then evaporated under reduced pressure to afford the crude solid, which was used for the next step without purification. [M+H]⁺ (m/z): 571.5.

### Step 4. 2-ammonioethyl (2-((5Z,5Z,11Z,14Z)-icosa-5,8,11,14-tetraenamido)ethyl) phosphate

To a stirred solution of 2-((tert-butoxycarbonyl)amino)ethyl (2-((5Z,8Z,11Z,14Z)-icosa-5,8,11,14-tetraenamido)ethyl) phosphate (48 mg, 0.084 mmol, 1.0 eq.) in DCM (2 mL) was added trifluoroacetic acid (TFA; 0.2 mL) at 0° C., then stirred at 25° C. for 2 h. After completion of the starting material, the reaction mixture was evaporated under reduced pressure to afford crude 2-ammonioethyl (2-((5Z,8Z,11Z,14Z)-icosa-5,8,11,14-tetraenamido)ethyl) phosphate (35 mg). The crude solid was purified by PREP-HPLC Method-B as an off-white solid (3.7 mg): LUNA-C18, 250×21.2 mm, 5 μm; Mobile phase: ACN:Water+HCOOH (0.1%); B %: 80%, 15 min 95%, 30 min. [M+H]⁺ (m/z): 471.4. ¹H-NMR (400 MHz, DMSO-d₆): δ 8.36 (s, 2H), 8.10 (s, 1H), 5.44-5.22 (m, 5H), 3.85-3.79 (m, 2H), 3.66-3.64 (m, 2H). 3.18 (q, J=5.6 Hz, 2H), 2.95 (s, 2H), 2.83-2.75 (m, 4H), 2.07-1.99 (m, 5H), 1.57-1.49 (m, 2H), 1.35-1.24 (m, 7H). 0.85 (t, J=6.8 Hz, 3H). ³¹P NMR (400 MHz, DMSO-d₆): δ 1.0

### Example 4. 2-ammonio-2-carboxyethyl (2-((5Z,8Z,11Z,14Z)-icosa-5,8,11,14-tetraenamido)ethyl) phosphate (Compound 5)

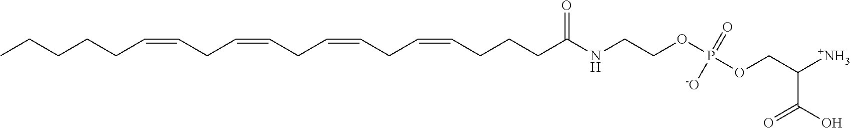

### Step 1. 2-(((benzyloxy)carbonyl)amino)ethyl (3-(tert-butoxy)-2-((tert-butoxycarbonyl)amino)-3-oxopropyl) phosphate

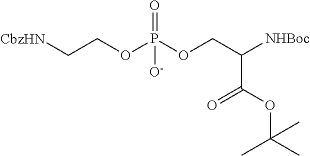 [SMILES: CC(C)(C)OC(=O)NC(COP(=O)([O-])OCCNC(=O)OCc1ccccc1)C(=O)OC(C)(C)C]

To a stirred solution of benzyl (2-hydroxyethyl)carbamate (1 g, 5.128 mmol, 1.0 eq.) in chloroform (20 mL) was added Et₃N (1.1 mL, 7.692 mmol, 1.5 eq.), followed by POCl₃ (0.52 mL, 5.641 mmol, 1.1 eq.) at −10° C. The reaction mixture was then stirred for 1 h at 25° C. Pyridine (3.6 mL, 44,102 mmol, 8.6 eq.) and Boc-L-Serine tert-butyl ester (2 g, 7.692 mmol, 1.5 eq) were added at −10° C. and the mixture was stirred at 25° C. for 18 h. The reaction mixture was then cooled to 0° C., and water was added (20 mL), The mixture was extracted with DCM (3×30 mL) and the organic layer was concentrated and purified by flash chromatography (Column: Biotage-CIB. 30 g Duo-100 Å 30 μm; Mobile phase: ACN:Water+0.015% NH₄HCO₃; min./B %: 0/0, 30/5, 35/20, 50/20, 55/100). Pure fractions were lyophilized to afford the title product (220 mg) as an off-white solid. [M−H]⁺ (m/z): 517.2; ¹H-NMR (400 MHz, DMSO-d₆): δ 7.94-7.89 (m, 1H), 7.64-7.58 (m, 1H), 7.39-7.27 (m, 5H), 7.09 (bs, 3H), 4.99 (s, 2H), 3.88-3.78 (m, 3H), 3.67-3.58 (m, 2H), 3.16-3.08 (m, 2H), 1.42-1.32 (m, 18H). ³¹P NMR (400 MHz, DMSO-d₆): 0.66.

### Step 2. 2-aminoethyl (3-(tert-butoxy)-2-((tertbutoxycarbonyl)amino)-3-oxopropyl) phosphate

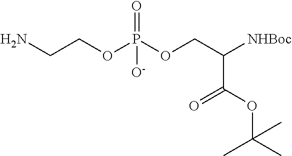 [SMILES: CC(C)(C)OC(=O)NC(COP(=O)([O-])OCCN)C(=O)OC(C)(C)C]

To a stirred solution of 2-(((benzyloxy)carbonyl)amino)ethyl (3-(tert-butoxy)-2-((tert-butoxycarbonyl)amino)-3-oxopropyl) phosphate (50 mg, 0.096 mmol, 1.0 eq.) in IP A. (2 mL) was added 10% Pd/C (50% wet) (15 mg) at 25° C. The reaction mixture was stirred under the atmosphere of hydrogen gas at 25° C. for 2 h. The reaction mixture was filtered through celite pad, washed with IP A (5 mL), and the filtrate was concentrated and dried under vacuum to afford the title compound (22 mg) as an off-white solid. [M+H]⁺ (m/z): 385.3; ¹H-NMR (400 MHz, DMSO-d₆): δ 8.23 (bs, 2H), 3.95-3.81 (m, 5H), 2.97-2.93 (m, 2H). 1.44-1.35 (m, 18H). ³¹P NMR (400 MHz, DMSO-d₆): 1.09.

### Step 3. 3˜(tert-butoxy)-2-((tert-butoxycarbonyl)amino)-3-oxopropyl (2-((5Z,8Z,11Z,14Z)-icosa-5,8,11,14-tetraenamido)ethyl) phosphate

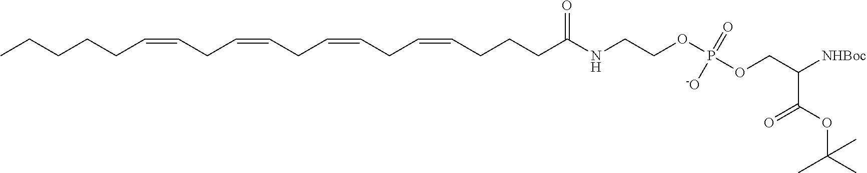

To a stirred solution of arachidonic acid (18 mg, 0.059 mmol, 1.0 eq.) in DMF (1 mL) was added HATU (26 mg, 0.071 mmol, 1.2 eq.) and stirred for 30 min. Next, DIPEA (0.03 mL, 0.177 mmol, 3.0 eq.) and 2-aminoethyl (3-(tert-butoxy)-2-((tertbutoxycarbonyl)amino)-3-oxopropyl) phosphate (22 mg, 0.059 mmol, 1.0 eq.) were added to the reaction mixture at 25° C. The resulting mixture was further stirred for 18 h at 25° C. The reaction mass was then concentrated under reduced pressure and the crude solid 3-(tert-butoxy)-2-((tert-butoxycarbonyl)amino)-3-oxopropyl (2-((5Z,8Z,11Z,14Z)-icosa-5,8,11,14-tetraenamido)ethyl) phosphate (63 mg) was used in the next step without any further purification. [M+H]⁺ (m/z) 671.5.

### Step 4. 2-ammonio-2-carboxyethyl (2-((5Z,8Z,11Z,14Z)-icosa-5,8,11,14-tetraenamido)ethyl) phosphate

To a stirred solution of 3-(tert-butoxy)-2-((tert-butoxycarbonyl)amino)-3-oxopropyl (2-((5Z,8Z,11Z,14Z)-icosa-5,8,11,14-tetraenamido)ethyl) phosphate (63 mg, 0.094 mmol, 1.0 eq.) in DCM (50 mL) WSLS added TFA (0.15 ml) at 0° C. and the mixture was stirred at 25° C. for 18 h. The reaction mixture was then concentrated under reduced pressure and the crude solid (40 mg) was purified via PREP-HPLC Method-C (Gemini 08.250×21.2×5 urn, 1.6 μm; Mobile phase: ACN/0.1% of formic acid in water; min./B %: 0/10, 15/70, 30/95). The resulting fractions were lyophilized to afford the title compound as an off-white solid (7 mg). [M+H]⁺ (m/z): 515.4; ¹H-NMR (400 MHz, DMSO-d₆): δ 8.05 (t, J=5.6 Hz, 1H), 5.39-5.29 (m, 8H), 4.09-4.03 (m, 2H), 3.70-3.64 (m, 2H), 3.18 (q, J=5.6 Hz, 2H), 2.84-2.74 (m, 6H), 2.10-1.99 (m, 6H), 1.55-1.51 (m, 2H), 1.35-1.24 (m, 6H), 0.85 (t, J=6.8 Hz, 3H). ³¹P NMR (400 MHz, DMSO-d₆): 0.18

### Example 5. 2-(((2-((5Z,8Z,11Z,14Z)-icosa-5,8,11,14-tetraenamido)ethoxy)carbonyl)oxy)ethyl (2-(trimethylammonio)ethyl) phosphate (Compound 6)

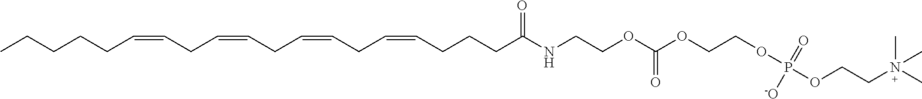

To a stirred solution of Compound 2 (Example 1; 100 mg, 0.2873 mmol, 1.0 eq.) in 6 mL of DCM at 0° C. was added pyridine (45 μL, 0.5747 mmol, 2.0 eq.), followed by 4-nitro phenyl chloroformate (69 mg, 0.3448 mmol, 1.2 eq.) and the reaction mixture was stirred at 25° C. for 6 h. The reaction mixture was then concentrated under vacuum to dryness. The obtained crude residue was dissolved in DMF (2 mL) and DMAP (45 mg, 0.3735 mmol, 1.3 eq.) was added, followed by Intermediate 1 (65 mg, 0.2873 mmol, 1.0 eq.) at 25° C. The reaction mixture was then stirred for 18 h at the same temperature. Then the reaction mixture was diluted with IPE (20 mL), and the solvent was decanted to obtain crude solid (130 mg), which was purified from according to PREP-HPLC Method-D: Gemini-C18, 250×21.2 mm, 5.0 μm; Mobile phase: (ACN:Water+HCOOH (0.1%)); B %: 30%, 20 min 75%, 25 min 95%) to afford the title compound as a pale brown solid (3.8 mg). [M+H]⁺ (m/z): 601.5; ¹H-NMR (400 MHz, DMSO-d₆): δ 8.22 (t, J=5.6 Hz, 1H), 5.39-5.27 (m, 8H), 4.16 (t, J=4.4 Hz, 2H), 4.07 (t, J=5.2 Hz, 2H). 4.05-3.98 (m, 2H), 3.84-3.77 (m, 2H), 3.49 (t, J=4.8, 2H), 3.31-3.26 (m, 2H), 3.12 (s, 9H), 2.83-2.74 (m, 6H), 2.08 (t, J=7.6, 2H), 2.05-1.97 (m, 4H), 1.57-1.49 (m, 2H), 1.35-1.22 (m, 6H), 0.85 (t, J=6.8 Hz, 3H). ³¹P-NMR (400 MHz, DMSO-d₆): δ −1.10

### Example 6. 3-(((2-((5Z,8Z,11Z,14Z)-icosa-5,8,11,14-tetraenamido)ethoxy)carbonyl)oxy)propyl (2-(trimethylammonio)ethyl) phosphate (Compound 7)

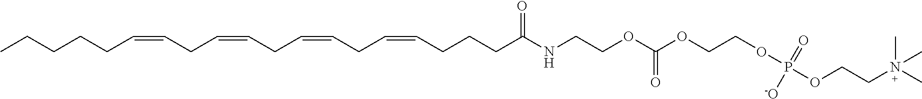

To a stirred solution of Compound 2 (Example 1; 100 mg, 0.287 mmol, 1.0 eq.) in 5 mL of DCM at 0° C. was added pyridine (46 μL, 0.574 mmol, 2.0 eq.) followed by 4-nitro phenyl chloroformate (69 mg, 0.344 mmol, 1.2 eq.), and the reaction was stirred at 25° C. for 6 h. The reaction mixture was then concentrated under vacuum to dryness. The obtained crude residue was dissolved in DMF (2 mL), and DMAP (45 mg, 0.373 mmol, 1.3 eq.) was added, followed by Intermediate 2 (69 mg, 0.287 mmol, 1.0 eq.) at 25° C., and stirred for 18 h at the same temperature. Then the reaction mixture was diluted with IPE (20 mL), and the solvent was decanted to obtain crude solid (150 mg), which was purified according to PREP-HPLC Method-E: Gemini-C18, 250×21.2 mm, 5.0 μm; Mobile phase: (ACN:Water+HCOOH (0.1%); B %: 70%, 20 min 20%) to afford the title compound as a pale brown solid (8 mg). [M+H]⁺ (m/z): 615.5; ¹H-NMR (400 MHz, DMSO-d₆): δ 8.24 (t, J=5.0 Hz, 1H), 5.37-5.31 (m, 8H), 4.13 (t, J=6.4 Hz, 2H), 4.06 (t, J=5.2 Hz, 2H), 4.01 (bs, 2H). 3.68 (q, J=6.4 Hz, 2H), 3.49 (t, J=4.4 Hz, 2H), 3.31-3.25 (m, 2H), 3.12 (s, 9H), 2.85-2.74 (m, 6H), 2.08 (t, J=7.2 Hz, 2H), 2.06-1.97 (m, 4H), 1.83-1.77 (m, 2H), 1.58-1.48 (m, 2H), 1.35-1.21 (m, 6H), 0.85 (t, J=6.8 Hz, 3H). ³¹P-NMR (400 MHz, DMSO-d₆): δ −0.90

### Example 7. 2-(((2-((5Z,8Z,11Z,14Z)-icosa-5,8,11,14-tetraenamido)ethoxy)carbonyl)amino)ethyl (2-(trimethylammonio)ethyl) phosphate (Compound 8)

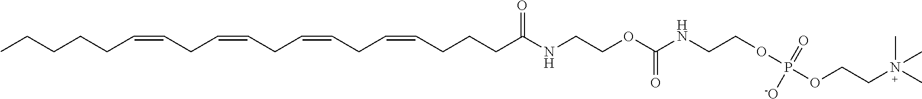

To a stirred solution of Compound 2 (Example 1; 100 mg, 0.287 mmol, 1.0 eq.) in 5 mL of DCM was added pyridine (46 μL, 0.574 mmol, 2.0 eq.) at 0° C., followed by 4-nitro phenyl chloroformate (69 mg, 0.344 mmol, 1.2 eq.). The mixture was stirred at 25° C. for 6 h and was concentrated under vacuum to dryness. The crude residue was dissolved in DMF (2 mL) and was added DMAP (45 mg, 0.373 mmol, 1.3 eq.) followed by Intermediate 3 (64 mg, 0.287 mmol, 1.0 eq.) at 25° C. and left to stir for 18 h at the same temperature. The reaction mixture was then diluted with IPE (20 mL), and the solvent was decanted to obtain crude solid (150 mg). The crude solid was purified according to PREP-HPLC Method-F: Gemini-C18, 250×21.2 mm, 5.0 μm; Mobile phase: (ACN:Water+HCOOH (0.1%); B %: 60%, 20 min 30%, 25 min 5%) to afford the title compound as a pale brown solid (10 mg). [M+H]⁺ (m/z): 600.5; ¹H-NMR (400 MHz, DMSO-d₆): δ 8.02 (t, J=5.6 Hz, 1H), 7.459 (t, J=5.2 Hz, 1H), 5.39-5.28 (m, 8H). 4.02 (bs, 2H), 3.91 (t, J=6.0 Hz, 2H), 3.69-3.62 (m, 2H). 3.52-3.47 (m, 2H), 3.21 (q, J=5.6 Hz, 2H), 3.12 (s, 9H), 3.11-3.07 (m, 2H). 2.83-2.74 (m, 6H), 2.11-1.97 (m, 6 h), 1.57-1.48 (m, 2H), 1.36-1.22 (m, 7H), 0.85 (t, J=6.8 Hz, 3H). ³¹P-NMR (400 MHz, DMSO-d₆): δ −0.31

The following compounds can be prepared using procedures similar to those described in Schemes 1-2 and/or the Examples provided herein, using appropriately substituted starting materials.

| Com- | |
|---|---|
| pound | |
| # | Compound Structure and Name |
| 9 | 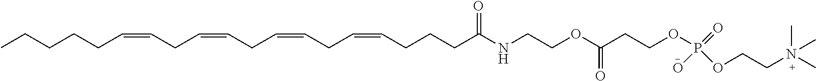 |
| | 3-(2-((5Z,8Z,11Z,14Z)-icosa-5,8,11,14-tetraenamido)ethoxy)-3-oxopropyl (2- |
| | (trimethylammonio)ethyl) phosphate |
| 10 | 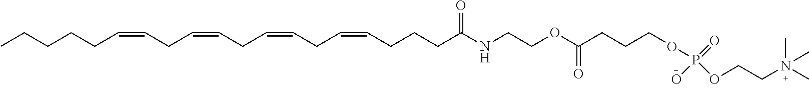 |
| | 4-(2-((5Z,8Z,11Z,14Z)-icosa-5,8,11,14-tetraenamido)ethoxy)-4-oxobutyl (2- |
| | (trimethylammonio)ethyl) phosphate |
| 11 | 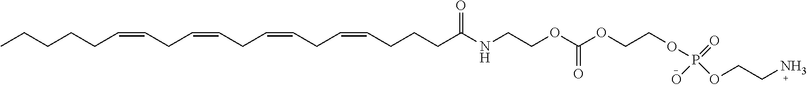 [SMILES: CCCCC/C=C\C/C=C\C/C=C\C/C=C\CCCC(=O)NCCOC(=O)OCCOP(=O)(O)OCCN] |
| | 2-ammonioethyl (2-(((2-((5Z,8Z,11Z,14Z)-icosa-5,8,11,14- |
| | tetraenamido)ethoxy)carbonyl)oxy)ethyl) phosphate |
| 12 | 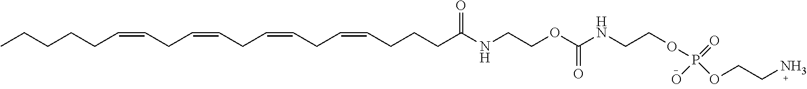 [SMILES: CCCCC/C=C\C/C=C\C/C=C\C/C=C\CCCC(=O)NCCOC(=O)NCCOP(=O)(O)OCCN] |
| | 2-ammonioethyl (2-(((2-((5Z,8Z,11Z,14Z)-icosa-5,8,11,14- |
| | tetraenamido)ethoxy)carbonyl)amino)ethyl) phosphate |
| 13 | 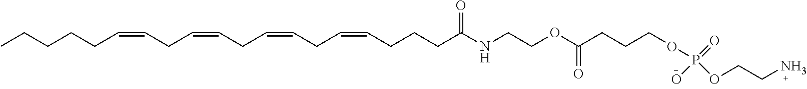 [SMILES: CCCCC/C=C\C/C=C\C/C=C\C/C=C\CCCC(=O)NCCOC(=O)CCCOP(=O)(O)OCCN] |
| | 2-ammonioethyl (4-(2-((5Z,8Z,11Z,14Z)-icosa-5,8,11,14-tetraenamido)ethoxy)-4- |
| | oxobutyl) phosphate |
| 14 | 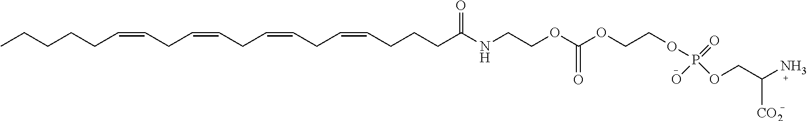 |
| | 2-ammonio-3-(((2-(((2-((5Z,8Z,11Z,14Z)-icosa-5,8,11,14- |
| | tetraenamido)ethoxy)carbonyl)oxy)ethoxy)oxidophosphoryl)oxy)propanoate |
| 15 | 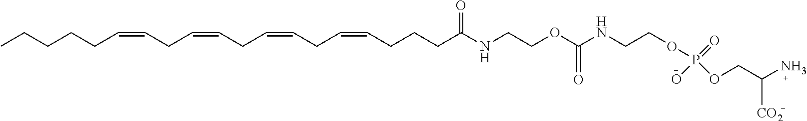 |
| | 2-ammonio-3-(((2-(((2-((5Z,8Z,11Z,14Z)-icosa-5,8,11,14- |
| | tetraenamido)ethoxy)carbonyl)amino)ethoxy)oxidophosphoryl)oxy)propanoate |
| 16 | 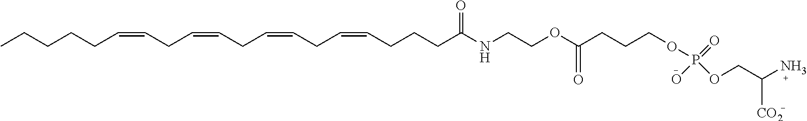 |
| | 2-ammonio-3-(((4-(2-((5Z,8Z,11Z,14Z)-icosa-5,8,11,14-tetraenamido)ethoxy)-4- |
| | oxobutoxy)oxidophosphoryl)oxy)propanoate |
| 17 | 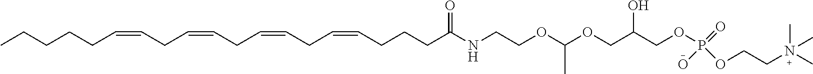 |
| | 2-hydroxy-3-(1-(2-((5Z,8Z,11Z,14Z)-icosa-5,8,11,14- |
| | tetraenamido)ethoxy)ethoxy)propyl (2-(trimethylammonio)ethyl) phosphate |
| 18 | 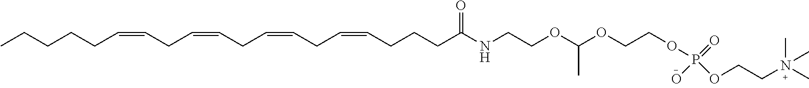 |
| | 2-(1-(2-((5Z,8Z,11Z,14Z)-icosa-5,8,11,14-tetraenamido)ethoxy)ethoxy)ethyl (2- |
| | (trimethylammonio)ethyl) phosphate |
| 19 | 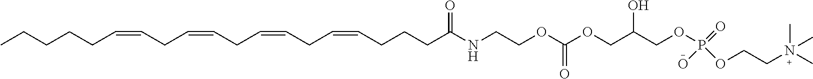 |
| | 2-hydroxy-3-(((2-((5Z,8Z,11Z,14Z)-icosa-5,8,11,14- |
| | tetraenamido)ethoxy)carbonyl)oxy)propyl (2-(trimethylammonio)ethyl) phosphate |
| 20 | 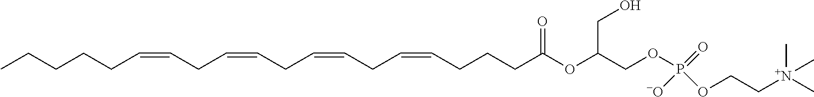 |
| | 2-hydroxy-2-(((5Z,8Z,11Z,14Z)-icosa-5,8,11,14-tetraenamido)oxy)propyl (2- |
| | (trimethylammonio)ethyl) phosphate |
| 21 | 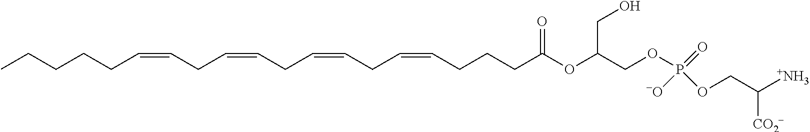 |
| | 2-ammonio-3-(((3-hydroxy-2-(((5Z,8Z,11Z,14Z)-icosa-5,8,11,14- |
| | tetraenoyl)oxy)propoxy)oxidophosphoryl)oxy)propanoate |
| 22 | 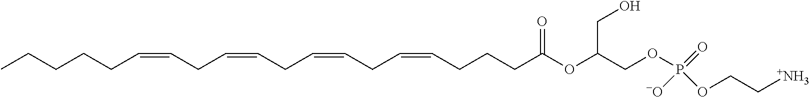 |
| | 2-ammonioethyl (3-hydroxy-2-(((5Z,8Z,11Z,14Z)-icosa-5,8,11,14- |
| | tetraenoyl)oxy)propyl) phosphate |
| 23 | 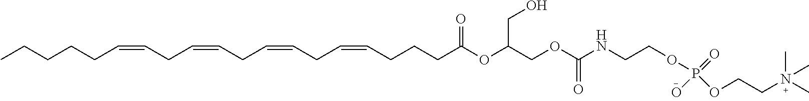 |
| | 2-(((3-hydroxy-2-(((5Z,8Z,11Z,14Z)-icosa-5,8,11,14- |
| | tetraenoyl)oxy)propoxy)carbonyl)amino)ethyl (2-trimethylammonio)ethyl) phosphate |
| 24 | 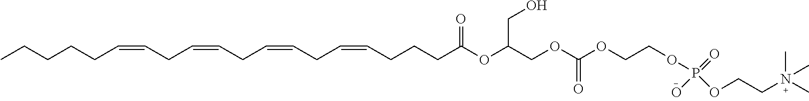 |
| | 2-(((3-hydroxy-2-(((5Z,8Z,11Z,14Z)-icosa-5,8,11,14- |
| | tetraenoyl)oxy)propoxy)carbonyl)oxy)ethyl (2-trimethylammonio)ethyl) phosphate |
| 25 | 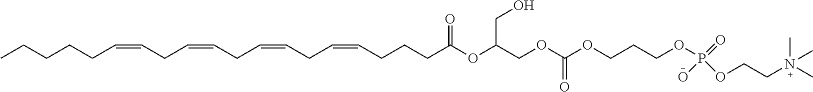 |
| | 3-(((3-hydroxy-2-(((5Z,8Z,11Z,14Z)-icosa-5,8,11,14- |
| | tetraenoyl)oxy)propoxy)carbonyl)oxy)propyl (2-trimethylammonio)ethyl) phosphate |
| 26 | 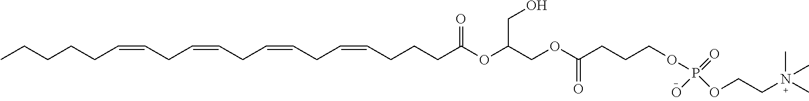 |
| | 4-(3-hydroxy-2-(((5Z,8Z,11Z,14Z)-icosa-5,8,11,14-tetraenoyl)oxy)propoxy)-4- |
| | oxobutyl (2-(trimethylammonio)ethyl) phosphate |
| 27 | 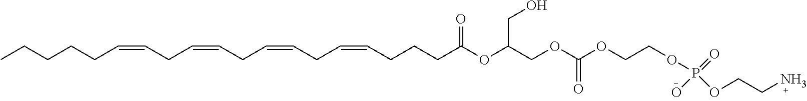 [SMILES: CCCCC/C=C\C/C=C\C/C=C\C/C=C\CCCC(=O)OC(CO)COC(=O)OCCOP(=O)(O)OCCN] |
| | 2-ammonioethyl (2-(((3-hydroxy-2-(((5Z,8Z,11Z,14Z)-icosa-5,8,11,14- |
| | tetraenoyl)oxy)propoxy)carbonyl)oxy)ethyl) phosphate |
| 28 | 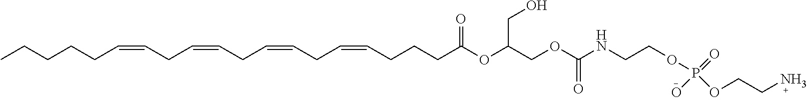 [SMILES: CCCCC/C=C\C/C=C\C/C=C\C/C=C\CCCC(=O)OC(CO)COC(=O)NCCOP(=O)(O)OCCN] |
| | 2-ammonioethyl (2-(((3-hydroxy-2-(((5Z,8Z,11Z,14Z)-icosa-5,8,11,14- |
| | tetraenoyl)oxy)propoxy)carbonyl)amino)ethyl) phosphate |
| 29 | 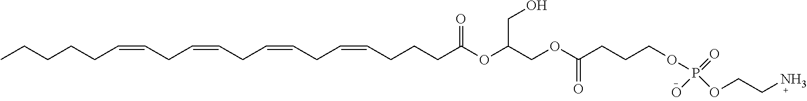 [SMILES: CCCCC/C=C\C/C=C\C/C=C\C/C=C\CCCC(=O)OC(CO)COC(=O)CCCOP(=O)(O)OCCN] |
| | 2-ammonioethyl (4-(3-hydroxy-2-(((5Z,8Z,11Z,14Z)-icosa-5,8,11,14- |
| | tetraenoyl)oxy)propoxy)-4-oxobutyl) phosphate |
| 30 | 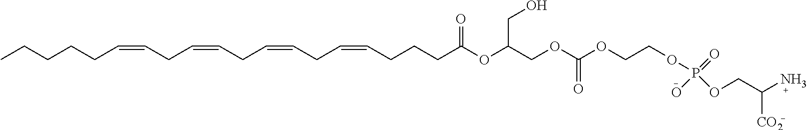 |
| | 2-ammonio-3-(((2-(((3-hydroxy-2-(((5Z,8Z,11Z,14Z)-icosa-5,8,11,14- |
| | tetraenoyl)oxy)propoxy)carbonyl)oxy)ethoxy)oxidophosphoryl)oxy)propanoate |
| 31 | 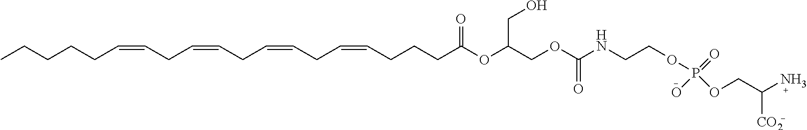 |
| | 2-ammonio-3-(((2-(((3-hydroxy-2-(((5Z,8Z,11Z,14Z)-icosa-5,8,11,14- |
| | tetraenoyl)oxy)propoxy)carbonyl)amino)ethoxy)oxidophosphoryl)oxy)propanoate |
| 32 | 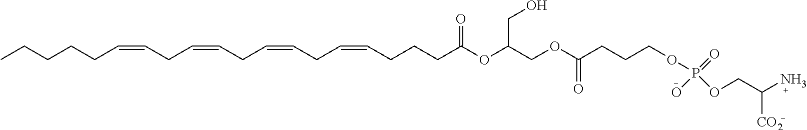 |
| | 2-ammonio-3-(((4-(3-hydroxy-2-(((5Z,8Z,11Z,14Z)-icosa-5,8,11,14- |
| | tetraenoyl)oxy)propoxy)-4-oxobutoxy)oxidophosphoryl)oxy)propanoate |
| 33 | 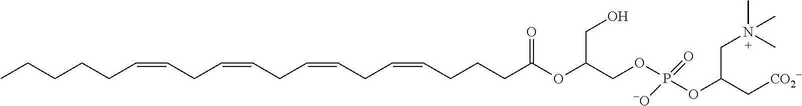 |
| | 3-(((3-hydroxy-2-(((5Z,8Z,11Z,14Z)-icosa-5,8,11,14- |
| | tetraenoyl)oxy)propoxy)oxidophosphoryl)oxy)-4-(trimethylammonio)butanoate |
| 34 | 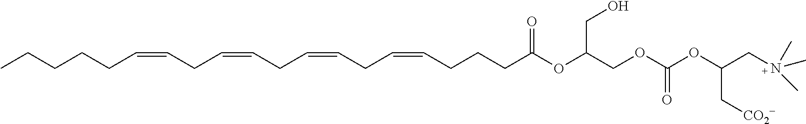 |
| | 3-(((3-hydroxy-2-(((5Z,8Z,11Z,14Z)-icosa-5,8,11,14- |
| | tetraenoyl)oxy)propoxy)carbonyl)oxy)-4-(trimethylammonio)butanoate |
| 35 | 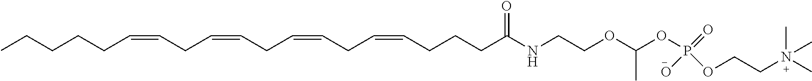 |
| | 1-(2-((5Z,8Z,11Z,14Z)-icosa-5,8,11,14-tetraenamido)ethoxy)ethyl (2- |
| | (trimethylammonio)ethyl) phosphate |
| 36 | 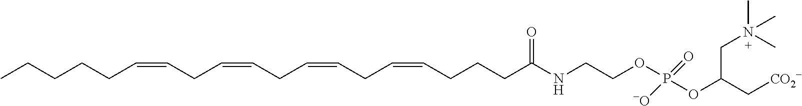 |
| | 3-(((2-((5Z,8Z,11Z,14Z)-icosa-5,8,11,14- |
| | tetraenamido)ethoxy)oxidophosphoryl)oxy)-4-(trimethylammonio)butanoate |
| 37 | 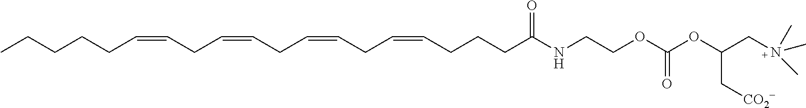 |
| | 3-(((2-((5Z,8Z,11Z,14Z)-icosa-5,8,11,14-tetraenamido)ethoxy)carbonyl)oxy)-4- |
| | (trimethylammonio)butanoate |
| 38 | 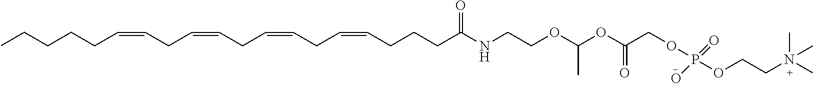 |
| | 2-(1-(2-((5Z,8Z,11Z,14Z)-icosa-5,8,11,14-tetraenamido)ethoxy)ethoxy)-2-oxoethyl |
| | (2-(trimethylammonio)ethyl) phosphate |

### Example 8. In vitro Mfsd2a Transport

### Methodology

The assay was conducted in a low throughput 6-well format with HEK293 cells prepared and then transfected in duplicate wells with plasmids containing either the wildtype (WT) version of hMfsd2a, the D97A mutant version, or an empty vector as control. Uptake into the ceils was assessed by both thin-layer chromatography (TLC) and ultrahigh performance liquid chromatography hyphenated mass spectrometry.

### Cell Transfection

HEK293 cells were seeded at 6.25×10⁵ per 6-well in 2 mL of DMEM with 10% FBS and 1% penicillin-streptomycin (P/S) media (Sigma) and incubated overnight at 37° C. in 5% CO₂. Cells were checked for confluency the next morning. On a per well basis the following lipid mix was generated, 6 μL of Lipofectamine 2000 was added dropwise to 200 μL of OptiMEM, this was left to stand for 5 minutes at room temperature (RT). 1 μg of hMfsd2a WT, D97A or empty plasmid was prepared in 200 μL of OptiMEM as appropriate for each well; the Lipofectamine 2000 in OptiMEM solution was then added dropwise to a total volume of 400 μL (this can be scaled to support the number of wells/plates to be assayed). This transfection preparation was then incubated at RT for 20 minutes. DMEM with 10% FBS and no P/S media was warmed to 37° C., the HEK293 plate media was changed and the cells washed carefully with 1 mL of the warmed DMEM with 10% FBS no P/S media, 1.6 mL of the warmed DMEM with 10% FBS no P/S media was then added to each well. 400 μL of the transfection preparation was then added dropwise to each well as appropriate and the plate was gently swirled in a circular motion. The plate was then incubated overnight at 37° C. in 5% CO₂.

Compound Incubation and Preparation of Analysis Samples Compound stock solutions were prepared in a 12% BSA in PBS solution such that a 40 μL spike into 2 ml, of plain DMEM would yield a concentration of 50 μM of test compound (the compound treated media). Remaining compound stock solution in 12% BSA in PBS was frozen at −20° C. to allow for media stability testing. The HEK293 6-well plate was removed from the incubator and the wells gently rinsed with 1 mL of plain DMEM that had been prewarmed to 37° C. 2 mL of the compound treated media was then added to each well. A 100 μL sample of the compound treated media was sampled to represent a T=0 h sample; 5 μL of this sample (the remainder was reserved and frozen in case of re-analysis being required) was diluted with 45 μL of DMEM and crashed with 50 μL MeCN in a 96-well plate which was sealed and kept on ice. The HEK293 6-well plate was then incubated at 37° C. in 5% CO₂ for 1 hour, the plate was then removed from the incubator and a 100 μL sample of media taken from each well to represent a T=1 h sample; 5 μL of this sample (the remainder was reserved and frozen in case of re-analysis being required) was diluted with 45 μL of DMEM and crashed with 50 μL MeCN into the 96-well plate which was re-sealed and stored at −20° C. The remaining media was then removed from the HEK293 6-well plate, the wells were gently rinsed twice with 1 mL of 0.5% BSA in DMEM, the media was then removed and the 6-well plate allowed to dry completely at RT. 1 ml of 3:2 Hexane:Isopropanol (HIP) was added to each well in a fume cupboard and the plate allowed to stand for 30 minutes at RT without shaking. The HIP solution was then transferred to 2 mL Eppendorf tubes, and the process was repeated with a second 1 mL aliquot of HIP and the two aliquots combined. The HIP samples were then dried down under a nitrogen stream.

### Thin Laver Chromatography (TLC) Analysis

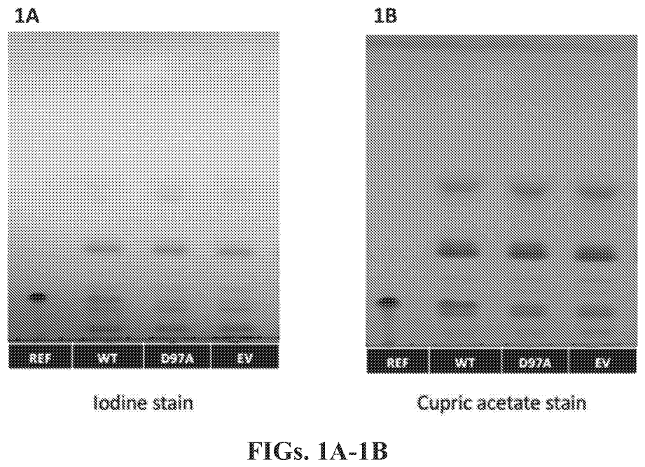

Silica gel plates were prepared in a fume cupboard by initially drawing a line 1.5 cm from the bottom edge of the plate and then drawing sample lanes with a width of 1 cm and a separation of 0.5 cm between lanes. TLC buffer for phospholipids was prepared as a 31:62:7 solvent mix of Methanol:Chloroform:Ammonium Hydroxide. Plates were pre-run in a humid chamber containing 200 mL of the TLC buffer until the solvent front was 1.5 cm from the plate edge, the plate was then allowed to dry. HIP samples prepared as described above were reconstituted in 50 μL of chloroform, briefly vortexed 3 times and then kept on ice. Samples were loaded onto the plate (along with reference compound) by streaking gently with a pipette tip, samples were allowed to dry between streaks. On completion of the sample loading the plate was run in a sealed humid chamber containing the TLC buffer as described above for approximately 1.5 hours or until the solvent front had nearly reached the top of the plate. The plate was removed from the chamber and dried. An initial image was taken using Bio-Rad Image lab 6.0. Iodine crystals were added to a new chamber which was sealed to allow the iodine vapor to saturate the container, the plate was then exposed to the iodine vapor in the chamber to allow visualization of bands of unsaturated fatty acids, once the plate was developed a second image was taken using Bio-Rad Image lab 6.0. The plate was then air-dried to remove the iodine. The plate was then saturated using a spray bottle with cupric acetate solution consisting of 3% cupric acetate by weight, 8% phosphoric acid by volume made up in an aqueous solution. The plate was allowed to dry for 5 minutes at RT and then heated in a fume cupboard using a hot air gun to make the bands more visible. A final image was acquired using the Bio-Rad Image lab 6.0. The difference in intensity between the bands generated from hMfsd2a (WT) or D97A transfected HEK293 cells were compared to the empty vector (EV) transfected cells, allowed for uptake into the cells driven by hMfsd2a to be identified against the reference (REF). Results of the TLC analysis are shown in FIGS. 1A-7B.

FIGS. 1A-1B show the TLC images from the iodine and cupric acetate stain respectively as described above with compound 3—lane from left: reference compound, HIP sample from WT cells, cells transfected with D97A mutant Mfsd2a and empty vector, with bands corresponding to the Compound 3 showing higher intensity in WT cells compared with cells transfected with D97A mutant Mfsd2a and/or empty vector, thus confirming the compound is transported via Mfsd2a.

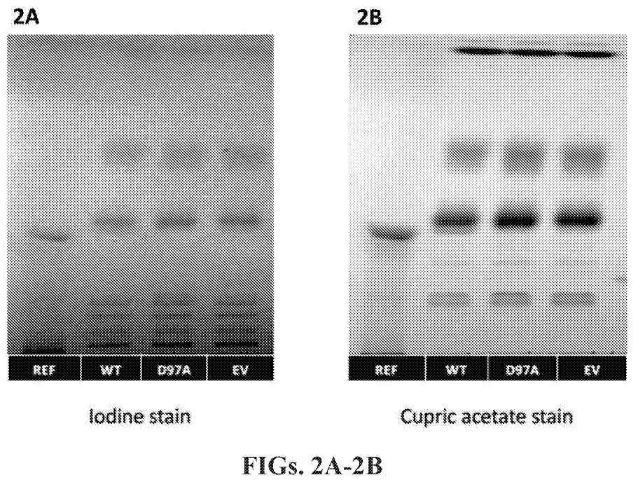

FIGS. 2A-2B correspond to TLC images as described above for compound 4, with bands corresponding to the Compound 4 showing higher intensity in WT cells compared with cells transfected with D97A mutant Mfsd2a and/or empty vector, thus confirming the compound is transported via Mfsd2a.

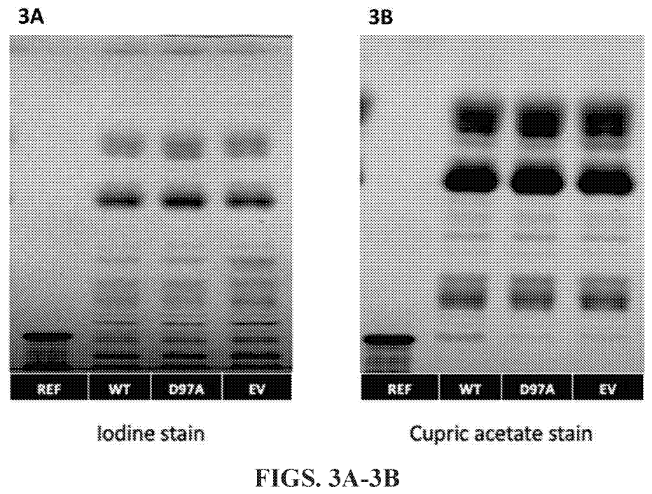

FIGS. 3A-3B correspond to TLC images as described above for compound 5, with bands corresponding to the Compound 5 showing higher intensity in WT cells compared with cells transfected with D97A mutant Mfsd2a and/or empty vector, thus confirming the compound is transported via Mfsd2a.

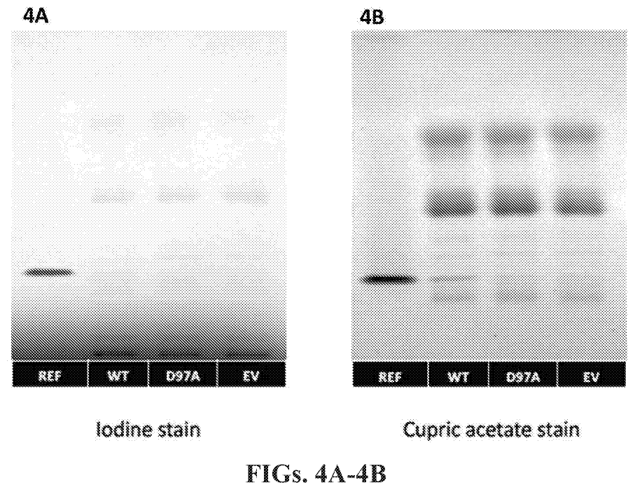

FIGS. 4A-4B correspond to TLC images as described above for compound 6, with bands corresponding to the Compound 6 showing higher intensity in WT cells compared with cells transfected with D97A mutant Mfsd2a and/or empty vector, thus confirming the compound is transported via Mfsd2a.

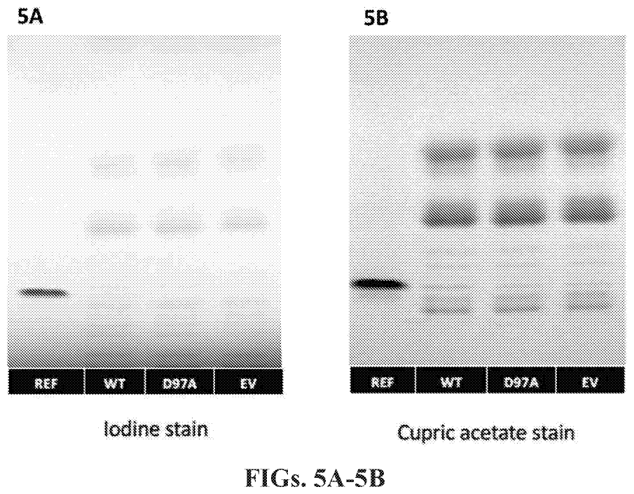

FIGS. 5A-5B correspond to TLC images as described above for compound 7, with bands corresponding to the Compound 7 showing higher intensity in WT cells compared with cells transfected with D97A mutant Mfsd2a and/or empty vector, thus confirming the compound is transported via Mfsd2a.

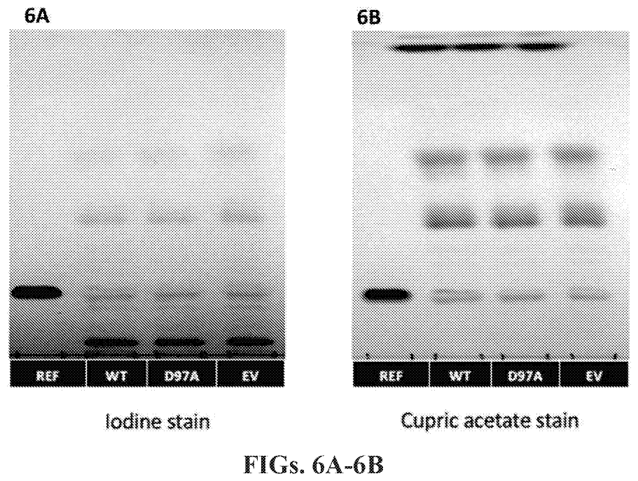

FIGS. 6A-6B correspond to TLC images as described above for compound 8, with bands corresponding to the Compound 8 showing higher intensity in WT cells compared with cells transfected with D97A mutant Mfsd2a and/or empty vector, thus confirming the compound is transported via Mfsd2a.

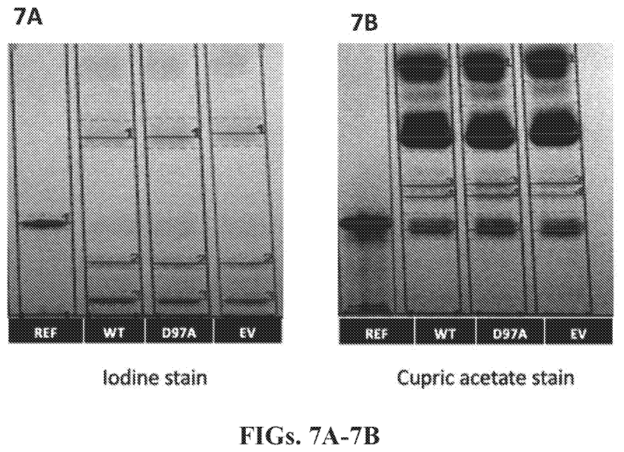

FIGS. 7A-7B correspond to TLC images as described above for compound 20, with bands corresponding to the Compound 20 where there is not significant differentiation of intensity in WT cells compared with cells transfected with D97A mutant Mfsd2a and/or empty vector. Hence, it cannot be confirmed if the compound is transported via Mfsd2a by this method.

### Example 9. In Vitro Mfsd2a Transport

### UPLC-MS-MS Analysis

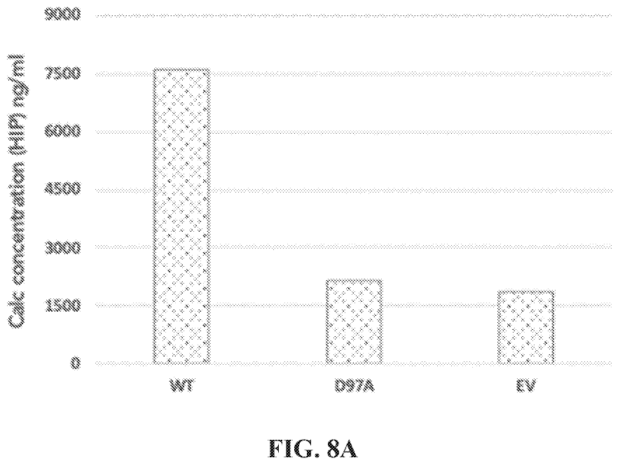

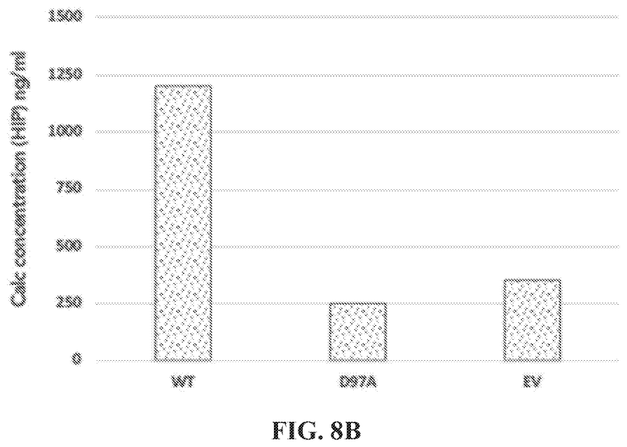

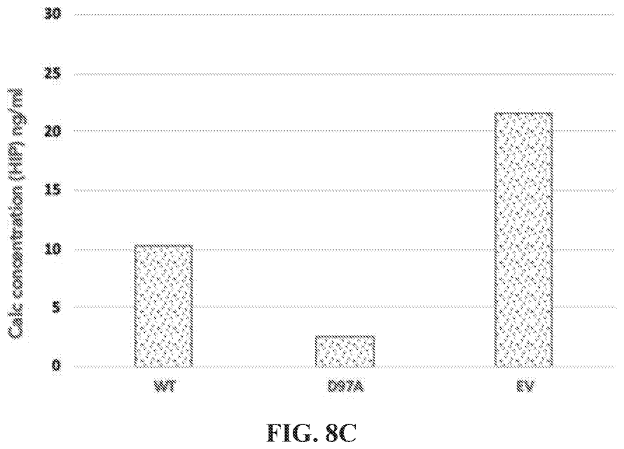

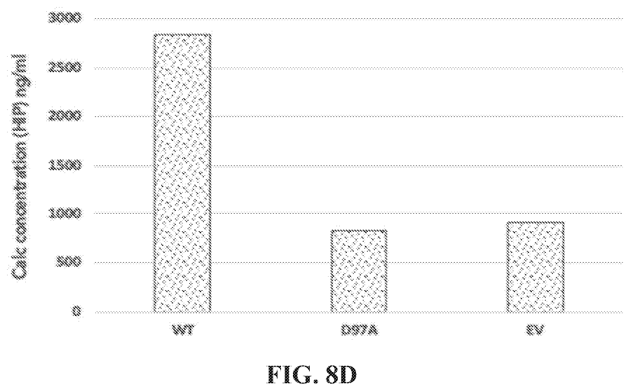

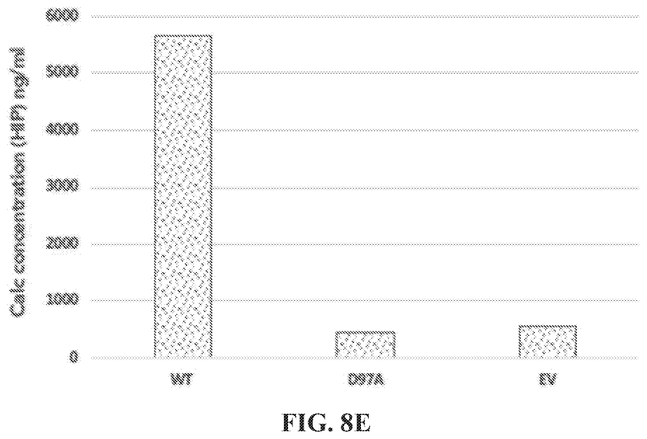

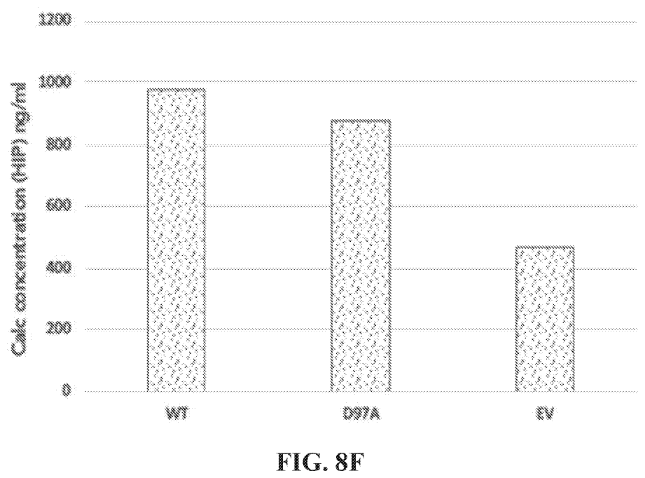

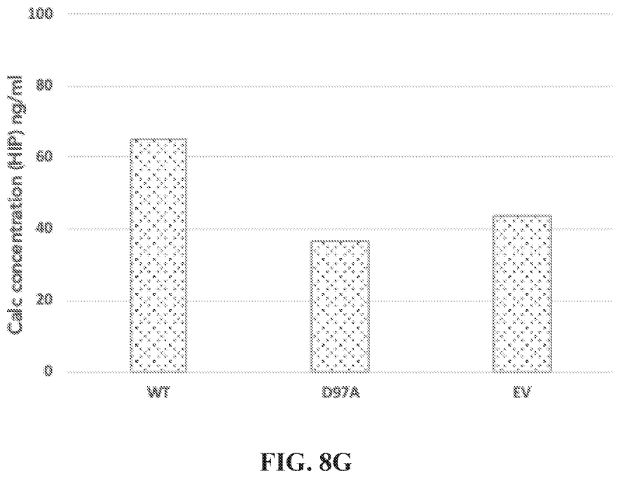

HIP samples prepared as described above were reconstituted in 100 μL of MeCN, vortex mixed and inverted multiple times to ensure all surfaces of the Eppendorf tube were rinsed with the MeCN and finally pulse centrifuged. A 50 μL aliquot of the MeCN reconstitution solution was then taken as a non-diluted HIP extract sample and added to the 96-well plate, alongside this a 1:10 dilution sample was prepared by taking a 5 μL aliquot and diluting with 45 μL of MeCN, 50 μL of Millipore water was added to each sample. A bioanalytical calibration line was prepared to cover a range of concentration from 0.0001 to 10 μM by spiking 2 μL of a 0.5 mM DMSO stock of the test compound into 98 μL of MeCN to generate a 10 μM top standard that was then serial diluted with MeCN to produce 6 calibration standard stocks. 50 μL of each calibration standard stock was added to the 96-well plate and diluted with 50 μL of Millipore water. 50 μL of an appropriate internal standard in MeCN was then added to each of the wells in the 96-well plate that contained either a sample or calibration standard, the plate was sealed and transferred to the UPLC-MS-MS system for analysis. Uptake of the test compound into the HEK293 cells determined from the HIP sample analysis with the impact of hMfsd2a assessed by comparing the ratio of the concentration of test compound in the hMfsd2a and D97A transfected cells to the empty vector transfected cells, as shown in FIGS. 8A-8G.

FIG. 8A shows the concentration of Compound 3 measured in HIP samples from WT cells, cells transfected with D97A mutant Mfsd2a and empty vector, with more Compound 3 detected in WT cells compared to cells transfected with D97A mutant Mfsd2a and/or empty vector, thus confirming the compound is transported via Mfsd2a.

FIG. 8B shows the concentration of compound 4 measured in HIP samples from WT cells, cells transfected with D97A mutant Mfsd2a and empty vector, with more Compound 4 detected in WT cells compared to cells transfected with D97A mutant Mfsd2a and/or empty vector, thus confirming the compound is transported via Mfsd2a.

FIG. 8C shows the concentration of compound 5 measured in HIP samples from WT cells, cells transfected with D97A mutant Mfsd2a and empty vector, with more Compound 5 detected in cells transfected with empty vector, compared with D97A mutant and/or WT. Hence, it cannot be confirmed if the compound is transported via Mfsd2a by this method.

FIG. 8D shows the concentration of compound 6 measured in HIP samples from WT cells, cells transfected with D97A mutant Mfsd2a and empty vector, with more Compound 6 detected in WT cells compared to cells transfected with D97A mutant Mfsd2a and/or empty vector, thus confirming the compound is transported via Mfsd2a.

FIG. 8E shows the concentration of compound 7 measured in HIP samples from WT cells, cells transfected with D97A mutant. Mfsd2a and empty vector, with more Compound 7 detected in WT cells compared to ceils transfected with D97A mutant Mfsd2a and/or empty vector, thus confirming the compound is transported via Mfsd2a.

FIG. 8F shows the concentration of compound 8 measured in HIP samples from WT cells, cells transfected with D97A mutant Mfsd2a and empty vector, with more Compound 8 detected in WT cells compared to cells transfected with D97A mutant Mfsd2a and/or empty vector, thus confirming the compound is transported via Mfsd2a.

FIG. 8G shows the concentration of compound 8 measured in HIP samples from WT cells, cells transfected with D97A mutant Mfsd2a and empty vector, with more Compound 20 detected in WT cells compared to cells transfected with D97A mutant Mfsd2a and/or empty vector, thus confirming the compound is transported via Mfsd2a.

### Example 10. In Vivo ADME

Protocol 1: IV JVC Rat PK Study at 3 mg/kg

Compound 3 was intravenously dosed at 3 mg/kg into a group of 3 individually housed, normally fed male Sprague Dawley rats that had been fitted with jugular vein catheters (JVCs). Dosing was performed with 2 mL/kg dosing volumes with 5% DMSO 95% water used as a dosing vehicle. Serial blood samples (150 μL) were taken from each animal at each time point post dose (0.08 h, 0.25 h, 0.5 h, 1 h, 2 h, 4 h, 8 h, and 24 h) into heparinised Eppendorf tubes (containing 5 μL heparin) on ice containing an equal quantity of water. Two 100 μL aliquots were then placed in duplicate 96 well plates on dry ice. Samples were stored in the freezer at −20° C. until bioanalysis.

Protocol 2: PO JVC Rat PK Study at 10 mg/kg

Compound 3 was orally dosed at 10 mg/kg to a group of 3 individually housed male Sprague Dawley rats that were fasted overnight and fed 4 h post-dose, the rats were fitted with JVCs. Dosing was performed with 5 mL/kg dosing volumes with 5% DMSO 95% water used as a dosing vehicle. Serial blood (150 μL) samples were taken from each animal at each timepoint (0.25 h, 0.5 h, 1 h, 2 h, 4 h, 6 h, 8 h, and 24 h) into heparinised Eppendorf tubes (containing 5 μL heparin) on ice containing an equal quantity of water. Two 100 μL aliquots were then placed in duplicate 96 well plates on dry ice. Samples were stored in the freezer at −20° C. until bioanalysis.

### Bioanalytical Samples Preparation

Samples were defrosted and acetonitrile (containing internal standard) was used to precipitate the proteins. All the samples were mixed, centrifuged and the supernatants analysed by LC-MS-MS according to the following procedures:
1. 20 μL plasma was transferred to a v-bottom 96-well plate.
2. 80 μL acetonitrile (ACN) containing IS was added to each well.
3. The plate was sealed and gently mixed for approximately 30 s.
4. Plates were centrifuged for 10 min at 3500 rpm.
5. 50 μL supernatant was transferred to a fresh v-bottom 96-well plate containing 100 μL MilliQ water.

Plates were thermo-sealed and stored at 4° C. until analysis. The resulting PK parameters are shown below in Table 1.

TABLE 1

| Dose | | Cₘₐₓ | Tₘₐₓ | CI | Vdss | t_{1/2} | AUC₀₋ₜ | AUC_{0-inf} | Bioavailability |
|---|---|---|---|---|---|---|---|---|---|
| (mg/kg) | Route | (ng/mL) | (h) | (mL/min/kg) | (L/kg) | (h) | (ng/mL*h) | (ng/mL*h) | (% F) |
| 3 | IV | — | — | 31 | 6.3 | 7.4 | 2462.2 | 2561.0 | — |
| 10 | PO | 372 | 2.5 | — | — | — | 2348.5 | 2426.6 | 28 |

Protocol 3: PO JVC Rat Terminal PK Study at 20 mg/kg

Compound 3 was orally dosed at 20 mg/kg to a group of nine, jugular vein cannulated, individually housed male Sprague Dawley rats that were fasted overnight and fed 4 h post-dose to assess its tissue distribution profile. Dosing was performed with 5 mL/kg dosing volumes with 5% DMSO, 95% water used as a dosing vehicle. Terminal blood samples (>230 μL) were taken from groups of 3 animals at each of 3 time-points post dose (1 h, 2 h, and 4 h) by cardiac puncture under CO₂ into heparinised Eppendorf tubes (containing 5 μL heparin) on ice containing an equal quantity of water. Two 100 μL aliquots were then placed in duplicate 96 well plates on dry ice. Immediately after blood sampling, brain and eye pair samples were excised, weighed, rinsed, blotted, and snap frozen in liquid N₂. Samples were stored in the freezer at −20° C. until bioanalysis.

Tissue samples were weighed and a volume of water 3 times the mass added to samples before homogenization. In the case of eyeballs the lens could not be homogenized, but all other tissues homogenized. The resulting data (tissue distribution parameters) are shown below in Table 2.

TABLE 2

| | | | | | Brain to |
|---|---|---|---|---|---|
| | Time | Blood | Brain | Eye | Blood Ratio |
| Compound | (h) | (ng/mL) | (ng/g) | (ng/g) | (corrected)* |
| Compound 3 | 1 | 108.4 | 28.3 | 1035.4 | 0.25 |
| | 2 | 16.7 | 28.5 | 105.0 | 1.69 |
| | 4 | 12.8 | 34.6 | 104.4 | 2.69 |

*Corrected for 15 μL of blood per gram of brain tissue contamination. Brown et al BR. J. Pharmac. (1986), 87, 569-578.

### Protocol 4, Brain Homogenate Analysis

The development of a more sensitive bioanalytical methodology for the detection of Compound 2 in brain tissues was required, as Compound 2 was not detected above the lower limit of quantification in the analysis above.

Re-analysis of the brain tissues harvested above (see Example 4) was conducted according to the following procedures and the data are shown in Table 3.
1. 100 μL of brain homogenate was transferred to a 2 mL square matrix deep well plate,
2. 300 μL acetonitrile (ACN) containing IS was dispensed on to the samples and mixed on a shaker.
3. The samples were centrifuged for 15 min at 3500 rpm.
4. 300 μL supernatant was transferred in to a fresh 2 mL square matrix deep well plate.
5. Samples were concentrated using nitrogen then reconstituted in 80 μL of 50:50 ACN:MilliQ water and shaken on a plate shaker.
6. Samples were then analyzed by LC-MS-MS.

TABLE 3

| Compound | Time (h) | Brain (ng/g) |
|---|---|---|
| Compound 2 | 1 | 526.21 |
| | 2 | 495.03 |
| | 4 | 548.75 |

### Example 11. In Vitro Brain Homogenate Stability

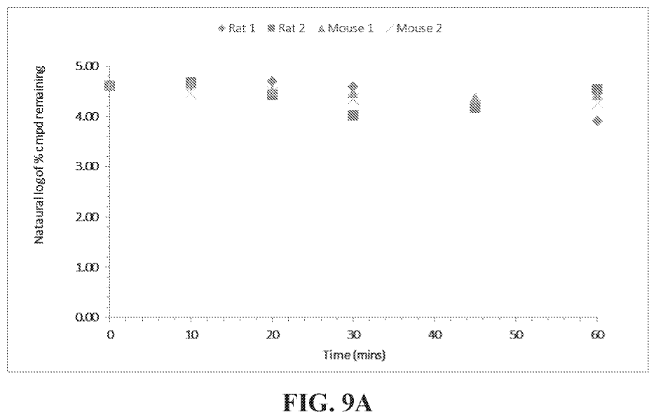

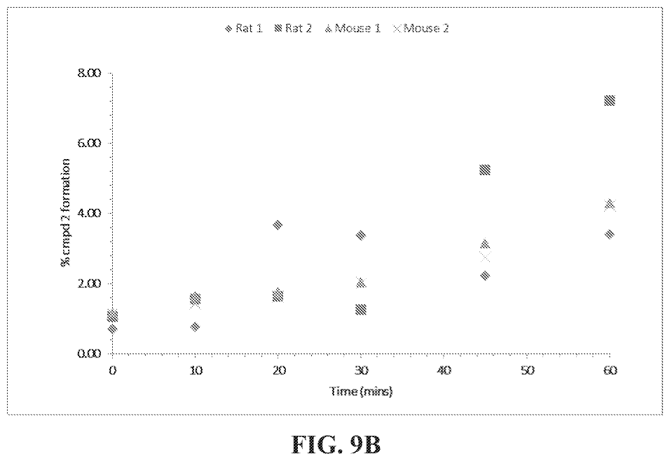

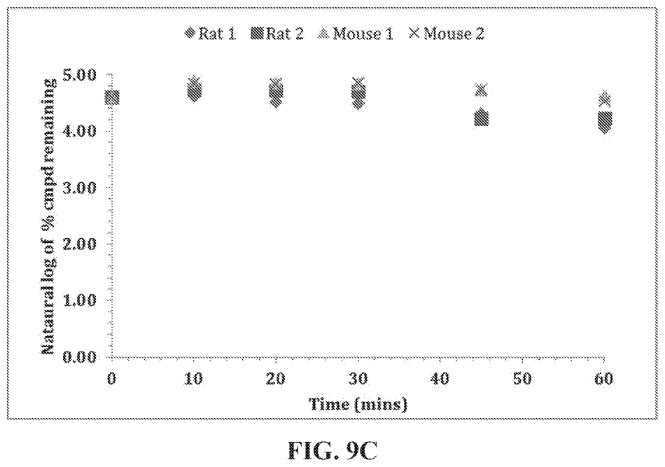

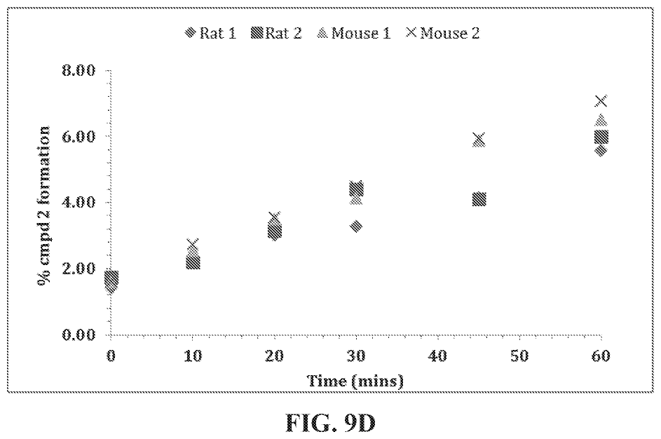

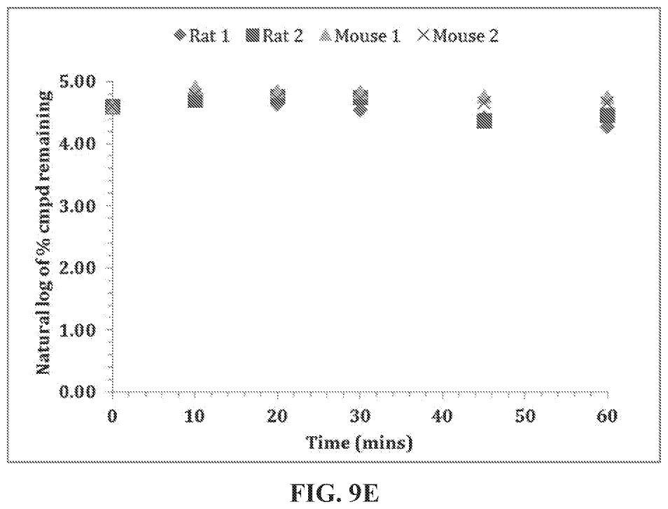

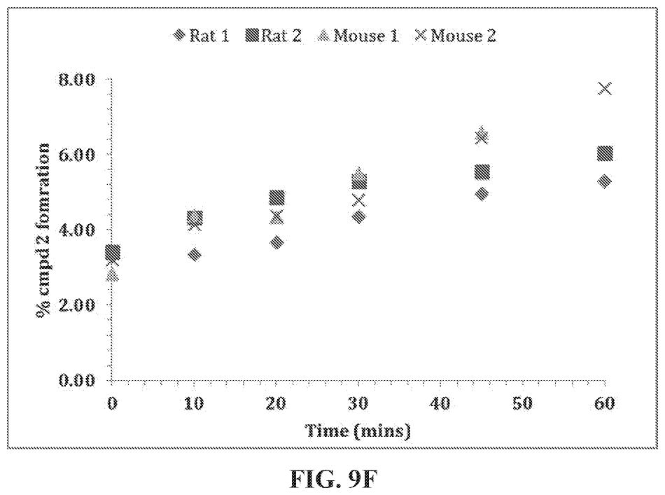

Fresh rodents brains were snap frozen immediately after collection and the assay performed on the same day. Brains were pre-incubated at 37° C. before being spiked with test compound at 1 μM. Samples were collected across 6 time points (0, 10, 20, 30, 45 and 60 minutes) and crashed in cold acetonitrile containing an internal standard (Leucine Enkephalin) and kept on ice until all samples had been collected. The samples were then shaken and centrifuged. 100 μL of supernatant was transferred to a plate containing 50 μL of MilliQ water and mixed. These plates were analyzed by LC-MS/MS over the 6 time points, where the percentage of test compound remaining was calculated using the peak area of the initial timepoint (T=0 minutes) as 100%. The natural log of the percentage remaining was then calculated. The natural log of the percentage remaining versus time (minutes) was plotted on a graph and the gradient of this graph was used to calculate the elimination rate constant (K), The half-life (t_{1/2}) of test compound was calculated using the natural log of 2 divided by the elimination rate constant. The percentage of Compound 2 formed is reported below in Tables 4-6. FIGS. 9A-9F show results of the brain homogenate stability conducted with representative compounds of the Examples.

TABLE 4

| | | Rat % | | Mouse % |
|---|---|---|---|---|
| | Rat t_{1/2} | Compound 2 | Mouse t_{1/2} | Compound 2 |
| Compound | (min) | (T = 60 min) | (min) | (T = 60 min) |
| Compound 3 | 56 | — | 100 | — |
| Compound 2 | — | 5.3 | — | 4.3 |

TABLE 5

| | | Rat % | | Mouse % |
|---|---|---|---|---|
| | Rat t_{1/2} | Compound 2 | Mouse t_{1/2} | Compound 2 |
| Compound | (min) | (T = 60 min) | (min) | (T = 60 min) |
| Compound 4 | 76 | — | 120 | — |
| Compound 2 | — | 5.8 | — | 6.8 |

TABLE 6

| | | Rat % | | Mouse % |
|---|---|---|---|---|
| | Rat t_{1/2} | Compound 2 | Mouse t_{1/2} | Compound 2 |
| Compound | (min) | (T = 60 min) | (min) | (T = 60 min) |
| Compound 5 | 126 | — | 258 | — |
| Compound 2 | — | 5.7 | — | 7.9 |

As shown in Table 4-6 and FIG. 9A-9F, Compounds 3, 4, 5 each demonstrated a level of clearance in both the rat and mouse brain homogenate stability study, with the increasing formation of the pharmacologically active Compound 2 (anandamide) over the one hour incubation period. These data are consistent with the in-vivo rat CNS penetration data using Compound 3, where Compound 2 was also detected in the brain.

### Bioanalytical Analysis

Bioanalytical samples were prepared according to the procedures described above for IX-MS-MS analysis. The samples were analyzed by LC-MSMS utilizing the AB Sciex QTRAP 5500. The instrument were set to operate in positive ion mode for all analyses and the data are shown below in Table 7.

TABLE 7

| | | | | | | Collision |
|---|---|---|---|---|---|---|
| | | | Declustering | Entrance | Collision | Cell Exit |
| | | | Voltage | Potential | Energy | Potential |
| Analyte | Parent | Daughter | (V) | (V) | (eV) | (CXP) |
| Compound 2 | 348.19 | 62 | 120 | 10 | 10 | 12 |
| Compound 3 | 513.26 | 183.98 | 120 | 10 | 20 | 12 |
| Compound 4 | 471.19 | 330.30 | 71 | 10 | 23 | 16 |
| Compound 5 | 515.20 | 330.20 | 1 | 10 | 23 | 18 |
| Compound 6 | 601.00 | 104.10 | 96 | 10 | 37 | 16 |
| Compound 7 | 615.25 | 242.20 | 101 | 10 | 39 | 12 |
| Compound 8 | 600.26 | 184.10 | 221 | 10 | 39 | 10 |
| Compound 20 | 544.29 | 184.10 | 151 | 10 | 31 | 16 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims. It should be appreciated by those persons having ordinary skill in the art(s) to which the present invention relates that any of the features described herein in respect of any particular aspect and/or embodiment of the present invention can be combined with one or more of any of the other features of any other aspects and/or embodiments of the present, invention described herein, with modifications as appropriate to ensure compatibility of the combinations. Such combinations are considered to be part of the present invention contemplated by this disclosure.

What is claimed is:

1. A method of treating a disease or disorder selected from the group consisting of pain, a pain-related disease or disorder, a mood disease or disorder, a disease or disorder of the central nervous system, an optical disease or disorder, cancer, a gastrointestinal disease or disorder, a renal disease or disorder, a renal-related disease or disorder, a cardiovascular disease or disorder, and a skin disease or disorder in a subject, comprising administering to the subject a therapeutically effective amount of a compound of Formula VI; 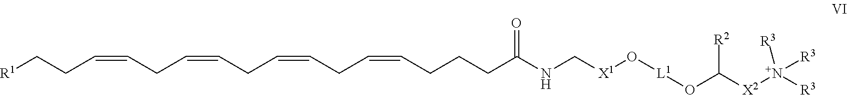
or a pharmaceutically acceptable salt thereof wherein:
L¹ is CO or PO₂;
X¹ is selected from the group consisting of C₁₋₄ alkylene, C₁₋₄ alkylene-OC(O)O—C₁₋₄ alkylene, C₁₋₄ alkylene-OC(O)C₁₋₄ alkylene, C₁₋₄ alkylene-O—C₁₋₄ alkylene, C₁₋₄ alkylene-O—C₁₋₄ alkylene-O—C₁₋₄ alkylene, C₁₋₄ alkylene-O—C₁₋₄ alkylene-O—C(O)C₁₋₄ alkylene, and C₁₋₄ alkylene-OC(O)NH—C₁₋₄ alkylene, wherein each C₁₋₄ alkylene is optionally substituted by OH or CO₂;
X² is C₁₋₆ alkylene, which is optionally substituted by OH or CO₂;
R¹ is C₁₋₁₀ alkyl,
R² is selected from the group consisting of H and C₁₋₆ alkyl, wherein the C₁₋₆ alkyl is optionally substituted by OH or CO₂; and
each R³ is independently selected from the group consisting of H and C₁₋₆ alkyl.

2. A method of treating a disease or disorder selected from the group consisting of pain, a pain-related disease or disorder, a mood disease or disorder, a disease or disorder of the central nervous system, an optical disease or disorder, cancer, a gastrointestinal disease or disorder, a renal disease or disorder, a renal-related disease or disorder, a cardiovascular disease or disorder, and a skin disease or disorder in a subject, comprising administering to the subject a therapeutically effective amount of a compound of Formula VII: 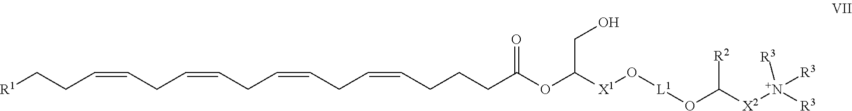
or a pharmaceutically acceptable salt thereof, wherein:
L¹ is CO or PO₂;
X¹ is selected from the group consisting of C₁₋₄ alkylene, C₁₋₄ alkylene-OC(O)O—C₁₋₄ alkylene, C₁₋₄ alkylene-OC(O)C₁₋₄ alkylene, C₁₋₄ alkylene-O—C₁₋₄ alkylene, C₁₋₄ alkylene-O—C₁₋₄ alkylene-O—C₁₋₄ alkylene, C₁₋₄ alkylene-O—C₁₋₄ alkylene-O—C(O)C₁₋₄ alkylene, and C₁₋₄ alkylene-OC(O)NH—C₁₋₄ alkylene, wherein each C₁₋₄ alkylene is optionally substituted by OH or CO₂;
X² is C₁₋₆ alkylene, which is optionally substituted by OH or CO₂,
R¹ is C₁₋₁₀ alkyl;
R² is selected from the group consisting of H and C₁₋₆ alkyl, wherein the C₁₋₆ alkyl is optionally substituted by OH or CO₂; and
each R³ is independently selected from the group consisting of H and C₁₋₆ alkyl.

3. The method of claim 1 or 2, wherein L¹ is CO.

4. The method of claim 1 or 2, wherein L¹ is PO₂.

5. The method of claim 1 or 2, wherein X¹ is selected from the group consisting of C₁₋₄ alkylene, C₁₋₄ alkylene-OC(O)O—C₁₋₄ alkylene, C₁₋₄ alkylene-OC(O)C₁₋₄ alkylene, C₁₋₄ alkylene-O—C₁₋₄ alkylene, C₁₋₄ alkylene-O—C₁₋₄ alkylene-O—C₁₋₄ alkylene, and C₁₋₄ alkylene-O—C₁₋₄ alkylene-O—C(O)C₁₋₄ alkylene, wherein each C₁₋₄ alkylene is optionally substituted by OH or CO₂.

6. The method of any one of claims 1 to 3, wherein X¹ is selected from the group consisting of CH₂, CH₂OC(O)O—C₁₋₄ alkylene, CH₂OC(O)C₁₋₄ alkylene, CH₂O—C₁₋₄ alkylene, CH₂O—C₁₋₄ alkylene-O—C₁₋₄ alkylene, CH₂—O—C₁₋₄ alkylene-O—C(O)C₁₋₄ alkylene, and C₁₋₄ alkylene-OC(O)NH—C₁₋₄ alkylene, wherein each C₁₋₄ alkylene is optionally substituted by OH.

7. The method of any one of claims 1 to 4, wherein X¹ is selected from the group consisting of CH₂, CH₂OC(O)O—C₁₋₄ alkylene, CH₂OC(O)C₁₋₄ alkylene, CH₂O—C₁₋₄ alkylene, CH₂O—C₁₋₄ alkylene-O—C₁₋₄ alkylene, and CH₂—O—C₁₋₄ alkylene-O—C(O)C₁₋₄ alkylene, wherein each C₁₋₄ alkylene is optionally substituted by OH.

8. The method of any one of claims 1 to 4, wherein X¹ is selected from the group consisting of CH₂, CH₂OC(O)OCH₂CH₂, CH₂OC(O)OCH₂CH(OH)CH₂, CH₂OC(O)CH₂CH₂CH₂, CH₂OC(O)CH₂CH₂, CH₂OCH(CH₃), CH₂OCH(CH₃)OCH₂CH(OH)CH₂, CH₂OCH(CH₃)OCH₂CH₂, CH₂OCH(CH₃)OC(O)CH₂, and CH₂OC(O)NHCH₂CH₂.

9. The method of any one of claims 1 to 4, wherein X¹ is CH₂.

10. The method of any one of claims 1 to 9, wherein X² is C₁₋₃ alkylene which is optionally substituted with OH or CO₂.

11. The method of any one of claims 1 to 9, wherein X² is CH₂ or CH₂CO₂.

12. The method of any one of claims 1 to 11, wherein R¹ is C₁₋₆ alkyl.

13. The method of any one of claims 1 to 11, wherein R¹ is propyl.

14. The method of any one of claims 1 to 13, wherein R² is selected from the group consisting of H and C₁₋₃ alkyl which is optionally substituted by OH or CO₂.

15. The method of any one of claims 1 to 13, wherein R² is H or CH₂CO₂.

16. The method of any one of claims 1 to 15, wherein each R³ is independently selected from the group consisting of H and C₁₋₃ alkyl.

17. The method of any one of claims 1 to 15, wherein each R³ is H.

18. The method of any one of claims 1 to 15, wherein each R³ is methyl or ethyl.

19. The method of claim 1 or 2, wherein:
L¹ is CO or PO₂;
X¹ is C₁₋₃ alkylene, which is optionally substituted by OH or CO₂;
X² is C₁₋₃ alkylene, which is optionally substituted by OH or CO₂,
R¹ is C₁₋₆ alkyl;
R² is selected from the group consisting of H and C₁₋₃ alkyl which is optionally substituted by OH or CO₂;
each R³ is independently selected from the group consisting of H and C₁₋₃ alkyl.

20. The method of claim 1 or 2, wherein;
L¹ is CO or PO₂;
X¹ is C₁₋₄ alkylene;
X² is C₁₋₃ alkylene, which is optionally substituted by OH or CO₂;
It is C₁₋₆ alkyl;
R² is selected from the group consisting of H and C₁₋₃ alkyl which is optionally substituted by CO₂;
each R³ is independently selected from the group consisting of H and C₁₋₃ alkyl.

21. The method of claim 1, wherein:
L¹ is PO₂;
X¹ is C₁₋₄ alkylene;
X² is C₁₋₃ alkylene, which is optionally substituted by OH or CO₂;
R¹ is C₁₋₆ alkyl;
R² is selected from the group consisting of H and C₁₋₃ alkyl which is optionally substituted by CO₂;
each R³ is independently selected from the group consisting of H and C₁₋₃ alkyl.

22. The method of claim 2, wherein:
L¹ is CO;
X¹ is C₁₋₄ alkylene;
X² is C₁₋₃ alkylene, which is optionally substituted by OH or CO₂;
R¹ is C₁₋₆ alkyl;
R² is selected from the group consisting of H and C₁₋₃ alkyl which is optionally substituted by CO₂;
each R³ is independently selected from the group consisting of H and C₁₋₃ alkyl.

23. The method of claim 1, wherein the compound of Formula VI is a compound of Formula II: 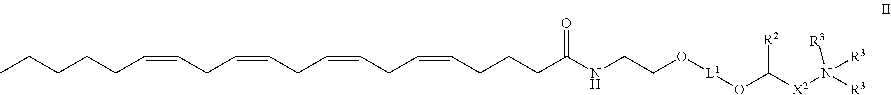
or a pharmaceutically acceptable salt thereof.

24. The method of claim 1, wherein the compound of Formula VI is a compound of Formula III: 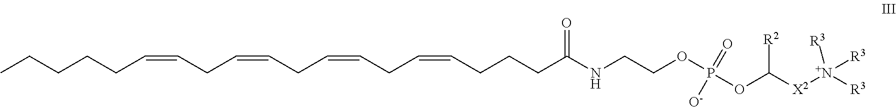
or a pharmaceutically acceptable salt thereof.

25. The method of claim 2, wherein the compound of Formula VII is a compound of Formula IV: 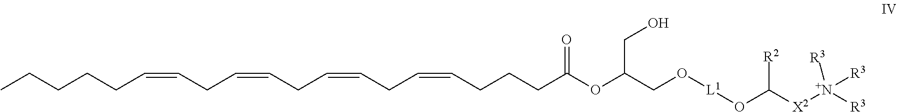
or a pharmaceutically acceptable salt thereof.

26. The method of claim 2, wherein the compound of Formula VII is a compound of Formula V: 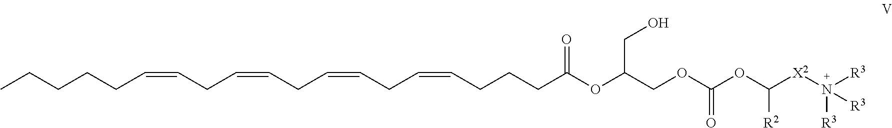
or a pharmaceutically acceptable salt thereof.

27. The method of claim 1, wherein the compound is selected from the group consisting of: 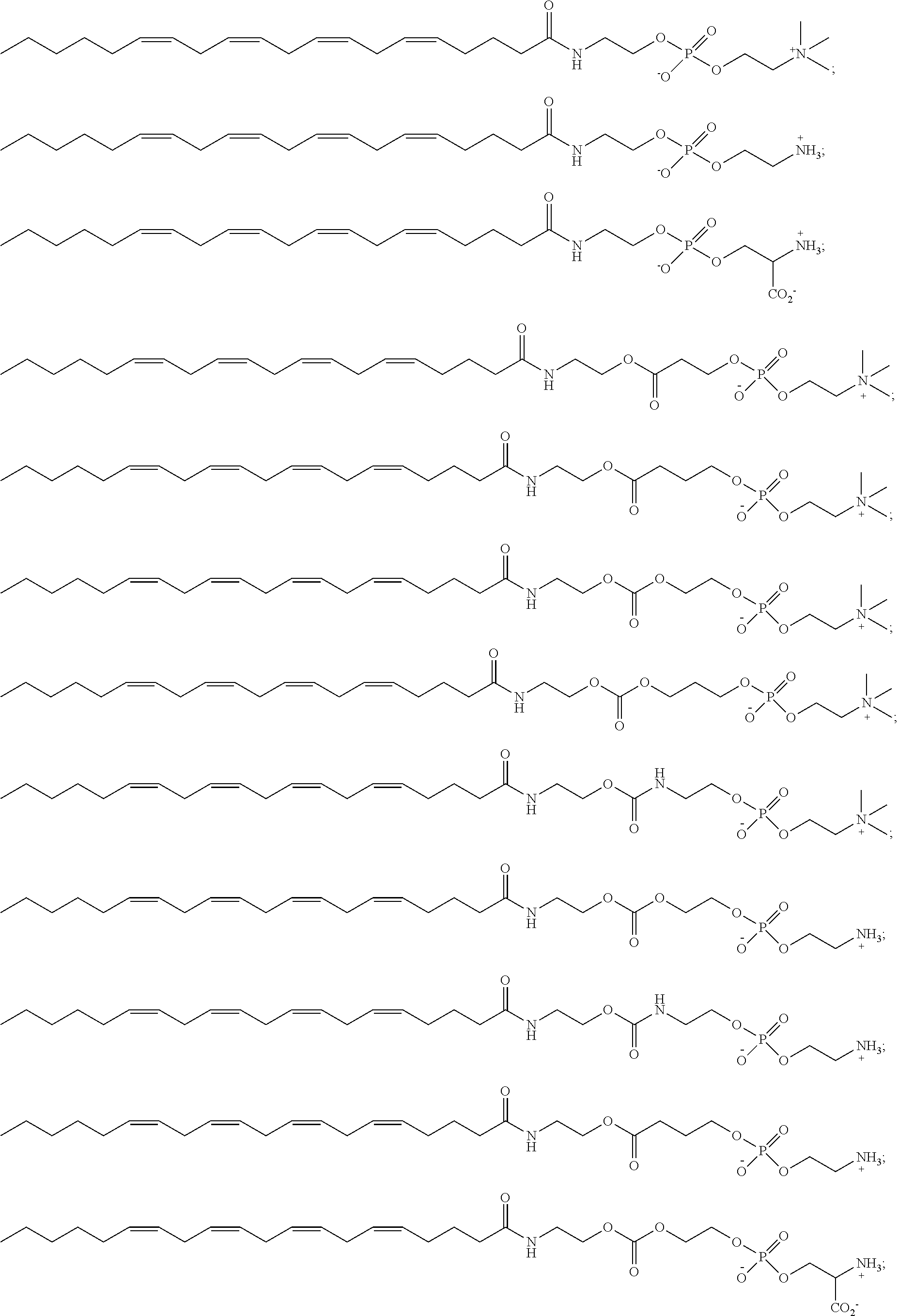 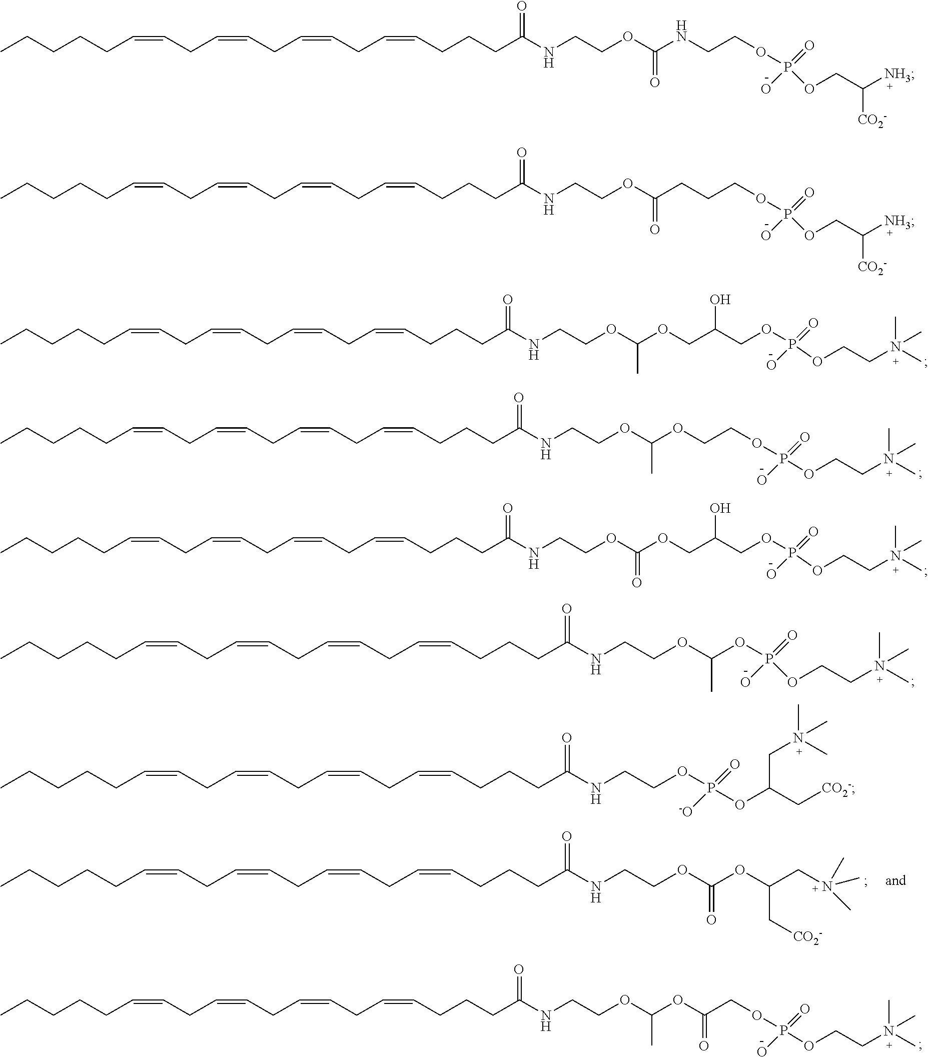
or a pharmaceutically acceptable salt thereof.

28. The method of claim 2, wherein the compound is selected from the group consisting of: 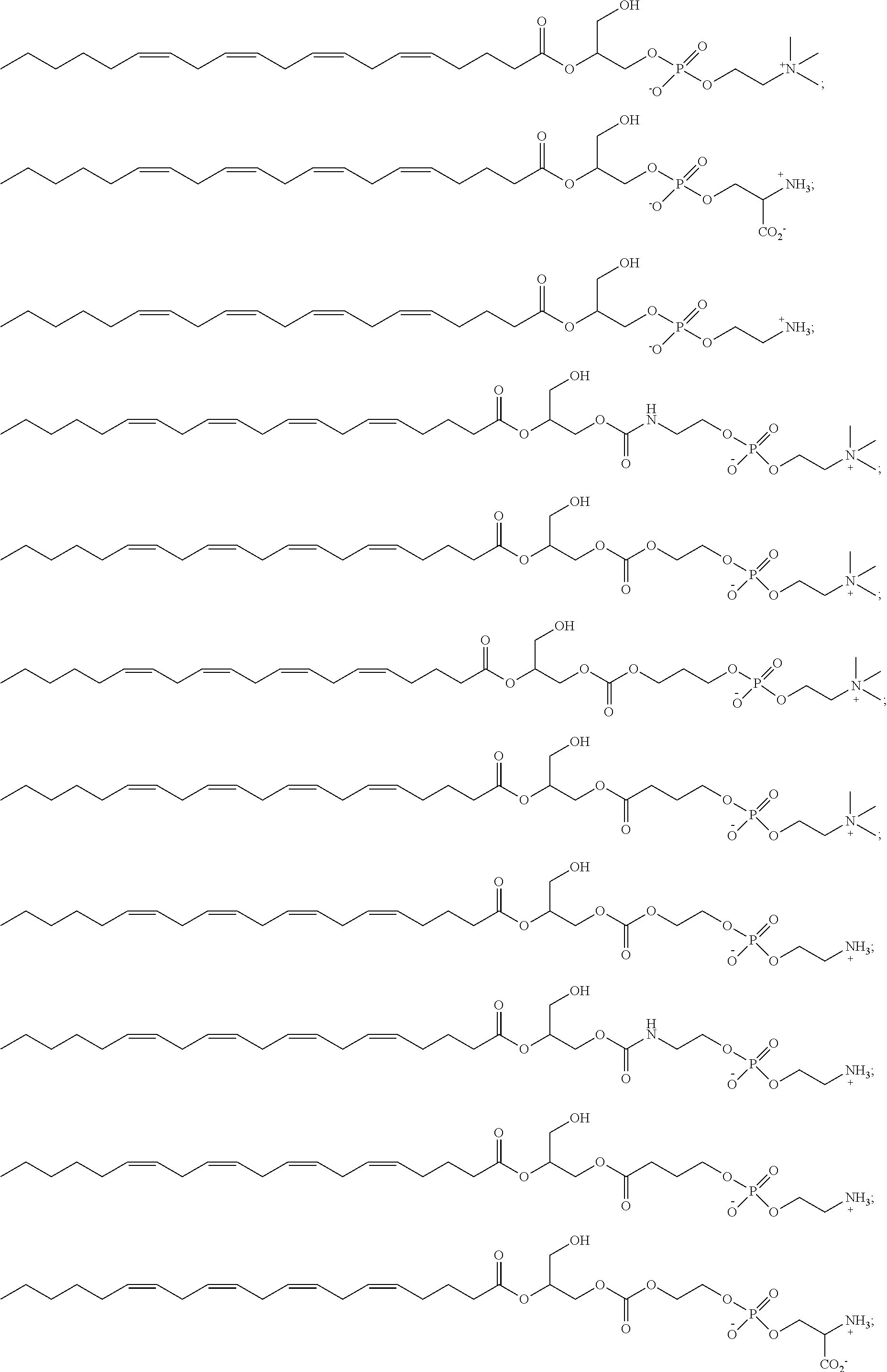 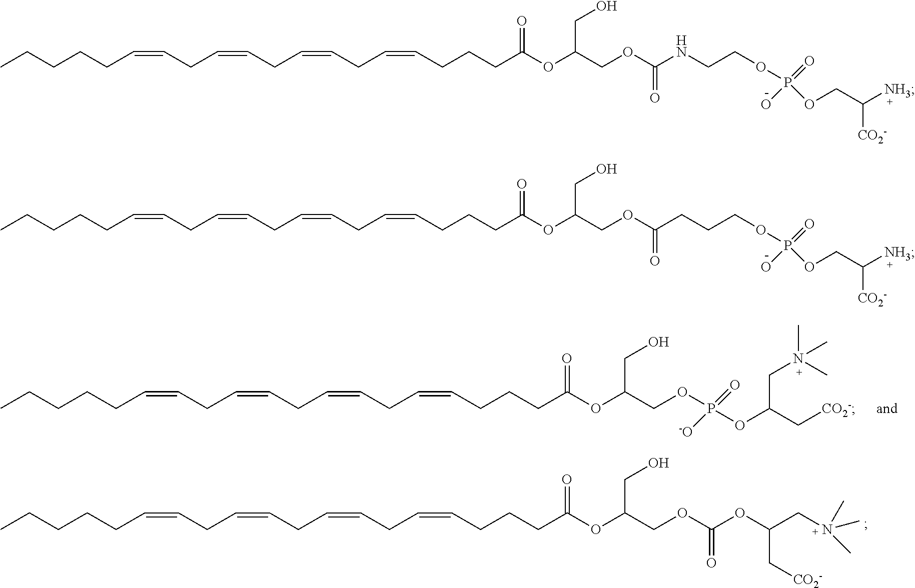
or a pharmaceutically acceptable salt thereof.

29. The method of claim 1, wherein the compound is: 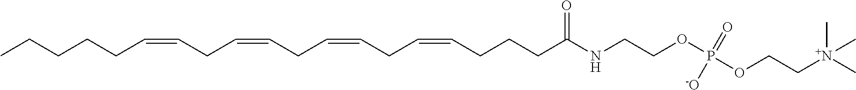
or a pharmaceutically acceptable salt thereof.

30. The method of any one of claims 1 to 29, wherein the compound is administered to the subject in the form of a pharmaceutical composition comprising the compound and one or more pharmaceutically acceptable excipients.

31. The method of any one of claims 1 to 30, wherein the disease or disorder is pain or a pain-related disease or disorder.

32. The method of claim 31, wherein the pain or a pain-related disease or disorder is selected from the group consisting of acute pain, chronic pain, neuropathic pain, nociceptive pain, inflammatory pain, cancer pain, fibromyalgia, rheumatoid arthritis, osteoarthritis, surgery-related pain, and osteoporosis.

33. The method of any one of claims 1 to 30, wherein the disease or disorder is a mood disease or disorder.

34. The method of claim 33, wherein the mood disease or disorder is selected from the group consisting of anxiety, depression, a sleeping disorder, an eating disorder, post-traumatic stress disorder, symptoms of drug or alcohol withdrawal, schizophrenia, obsessive-compulsive disorder, bipolar disorder, sexual dysfunction, attention deficit disorder (ADD), and attention deficit, hyperactivity disorder (ADHD).

35. The method of any one of claims 1 to 30, wherein the disease or disorder is a disease or disorder of the central nervous system or an optical disease or disorder.

36. The method of claim 35, wherein the disease or disorder of the central nervous system or an optical disease or disorder is selected from the group consisting of a demyelinating disease, glaucoma, age-related macular degeneration (AMD), amyotrophic lateral sclerosis (ALS), a cognitive disorder, Alzheimer's disease, a movement disorder, Huntington's chorea, Tourette's syndrome, Niemann-Pick disease, Parkinson's disease, epilepsy, a cerebrovascular disorder, and brain injury.

37. The method of claim 36, wherein the demyelinating disease is selected from the group consisting of multiple sclerosis (MS), neuromyelitis optica (NMO), Devic's disease, central nervous system neuropathy, central pontine myelinolysis, syphilitic myelopathy, leukoencephalopathies, leukodystrophies, Guillain-Barre syndrome, chronic inflammatory demyelinating polyneuropathy, anti-myelin-associated glycoprotein (MAG) peripheral neuropathy, Charcot-Marie-Tooth disease, peripheral neuropathy, myelopathy, optic neuropathy, progressive inflammatory neuropathy, optic neuritis, and transverse myelitis.

38. The method of any one of claims 1 to 30, wherein the disease or disorder is cancer.

39. The method of claim 38, wherein the cancer is selected from the group consisting of leukemia, mantle cell lymphoma, Hodgkin lymphoma, Non-Hodgkin lymphoma, hepatocellular carcinoma, ovarian cancer, colorectal cancer, pancreatic cancer, prostate cancer, breast cancer, glioma, skin cancer, renal carcinoma and lung cancer

40. The method of any one of claims 1 to 30, wherein the disease or disorder is a gastrointestinal disease or disorder.

41. The method of claim 40, wherein the gastrointestinal disease or disorder is selected from the group consisting of inflammatory bowel disease, gastroesophageal reflux disease, paralytic ileus, secretory diarrhoea, gastric ulcer, nausea, emesis, and a liver disorder.

42. The method of claim 41, wherein the liver disease is selected from the group consisting of acute liver failure, Alagille syndrome, hepatitis, enlarged liver, Gilbert's syndrome, liver cyst, liver haemangioma, fatty liver disease, steatohepatitis, primary sclerosing cholangitis, fascioliasis, primary bilary cirrhosis, Budd-Chiari syndrome, hemochromatosis, Wilson's disease, and transthyretin-related hereditary amyloidosis.

43. The method of any one of claims 1 to 30, wherein the disease or disorder is a renal disease or disorder or a renal-related disease or disorder.

44. The method of claim 43, wherein the renal disease or disorder or a renal-related disease or disorder is selected from the group consisting of diabetes, diabetic nephropathy, acute inflammatory kidney injury, renal ischemia urinary incontinence, and overactive bladder.

45. The method of any one of claims 1 to 30, wherein the disease or disorder is a skin disease or disorder.

46. The method of claim 45, wherein the skin disease or disorder is psoriasis or lupus.

47. The method of any one of claims 1 to 30, wherein the disease or disorder is a cardiovascular disease or disorder.

48. The method of claim 47, wherein the cardiovascular disease or disorder is selected from the group consisting of cardiovascular disease, vascular inflammation, idiopathic pulmonary fibrosis, and hypertension.

49. The method of any one of claims 30 to 48, wherein the compound is a compound of Formula VIa: 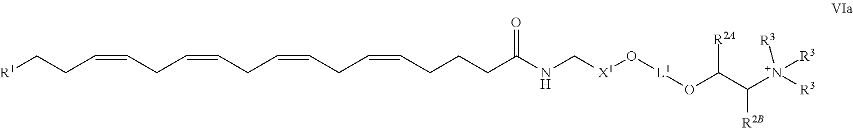
or a pharmaceutically acceptable salt thereof, wherein:
L¹ is CO or PO₂;
X¹ is selected from the group consisting of C₁₋₄ alkylene, C₁₋₄ alkylene-OC(O)O—C₁₋₄ alkylene, C₁₋₄ alkylene-OC(O)C₁₋₄ alkylene, C₁₋₄ alkylene-O—C₁₋₄ alkylene, C₁₋₄ alkylene-O—C₁₋₄ alkylene-O—C₁₋₄ alkylene, C₁₋₄ alkylene-O—C₁₋₄ alkylene-O—C(O)C₁₋₄ alkylene, and C₁₋₄ alkylene-OC(O)NH—C₁₋₄ alkylene, wherein each C₁₋₄ alkylene is optionally substituted by OH or CO₂;
X² is C₁₋₆ alkylene, which is optionally substituted by OH or CO₂;
R¹ is C₁₋₁₀ alkyl;
R^{2A} is H or CH₂CO₂;
R^{2B} is H or CO₂; and
each R³ is independently selected from the group consisting of H and C₁₋₆ alkyl.

50. The method of any one of claims 30 to 48, wherein the compound of Formula VII is a compound of Formula VIIa: 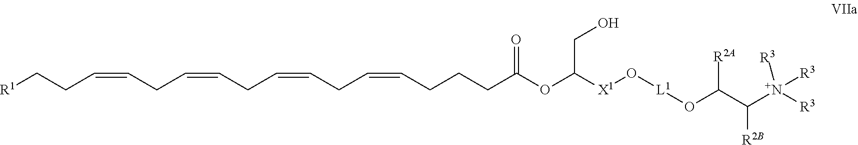
or a pharmaceutically acceptable salt thereof, wherein:
L¹ is CO or PO₂;
X¹ is selected from the group consisting of C₁₋₄ alkylene, C₁₋₄ alkylene-OC(O)O—C₁₋₄ alkylene, C₁₋₄ alkylene-OC(O)C₁₋₄ alkylene, C₁₋₄ alkylene-O—C₁₋₄ alkylene, C₁₋₄ alkylene-O—C₁₋₄ alkylene-O—C₁₋₄ alkylene, C₁₋₄ alkylene-O—C₁₋₄ alkylene-O—C(O)C₁₋₄ alkylene, and C₁₋₄ alkylene-OC(O)NH—C₁₋₄ alkylene, wherein each C₁₋₄ alkylene is optionally substituted by OH or CO₂;
X² is C₁₋₄ alkylene, which is optionally substituted by OH or CO₂;
R¹ is C₁₋₁₀ alkyl;
R^{2A} is H or CH₂CO₂;
R^{2B} is H or CO₂; and
each R³ is independently selected from the group consisting of H and C₁₋₆ alkyl.

51. A compound of Formula VIa: 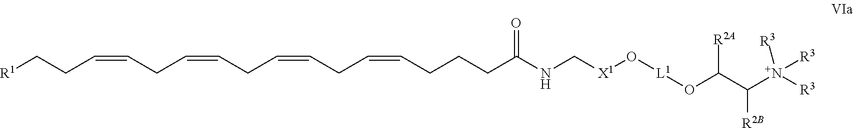
or a pharmaceutically acceptable salt thereof, wherein:
L¹ is CO or PO₂;
X¹ is selected from the group consisting of C₁₋₄ alkylene, C₁₋₄ alkylene-OC(O)O—C₁₋₄ alkylene, C₁₋₄ alkylene-OC(O)C₁₋₄ alkylene, C₁₋₄ alkylene-O—C₁₋₄ alkylene, C₁₋₄ alkylene-O—C₁₋₄ alkylene-O—C₁₋₄ alkylene, C₁₋₄ alkylene-O—C₁₋₄ alkylene-O—C(O)C₁₋₄ alkylene, and C₁₋₄ alkylene-OC(O)NH—C₁₋₄ alkylene, wherein each C₁₋₄ alkylene is optionally substituted by OH or CO₂,
X² is C₁₋₄ alkylene, which is optionally substituted by OH or CO₂;
R¹ is C₁₋₁₀ alkyl;
R^{2A} is H or CH₂CO₂,
R^{2B} is H or CO₂; and
each R³ is independently selected from the group consisting of H and C₁₋₆ alkyl.

52. The compound of claim 51, selected from the group consisting of: 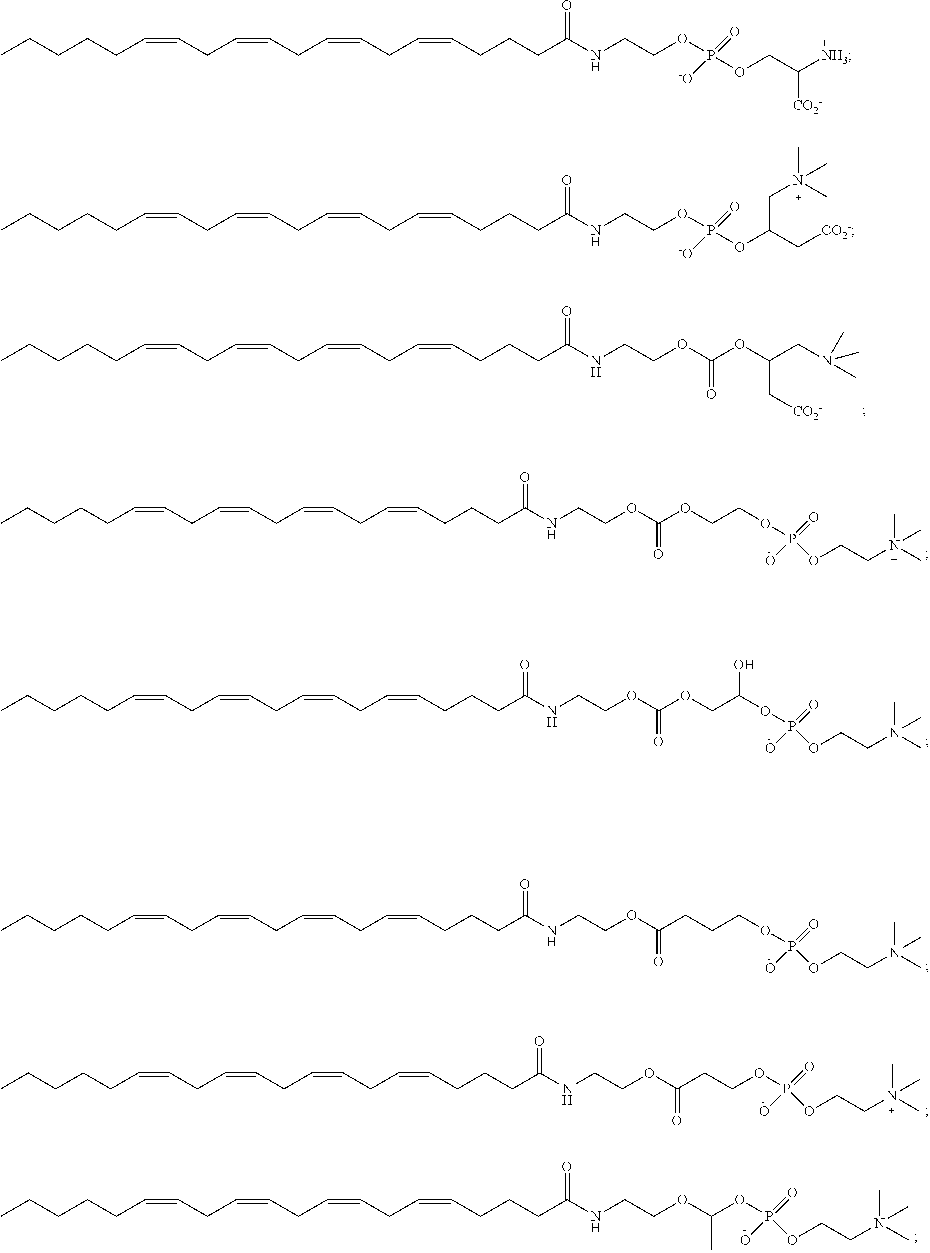 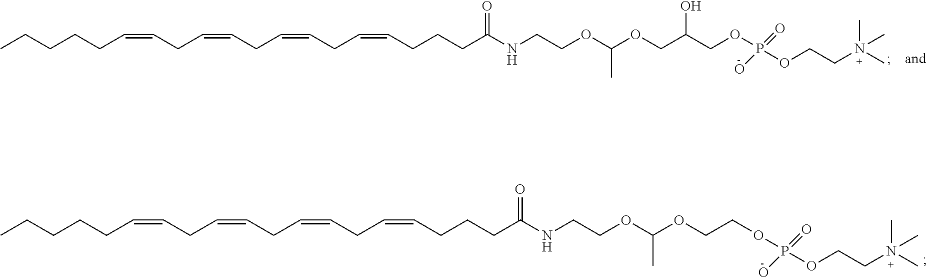
or a pharmaceutically acceptable salt thereof.

53. A pharmaceutical composition comprising a compound of claim 51 or 52, or a pharmaceutically acceptable salt thereof and one or more pharmaceutically acceptable excipients.
